(19) United States
(12) Patent Application Publication
(10) Pub. No.: US 2023/0053493 A1
(43) Pub. Date: Feb. 23, 2023

(54) ONE STEP METHODS, KITS, AND SYSTEMS FOR THE MEASUREMENT OF CONCENTRATIONS OF UNBOUND BILIRUBIN IN BIOLOGICAL FLUIDS

(71) Applicant: Alan Marc Kleinfeld, La Jolla, CA (US)

(72) Inventors: Alan Marc Kleinfeld, La Jolla, CA (US); Andrew H. Huber, La Jolla, CA (US); Hiroto Kameyama, La Jolla, CA (US); Tung-Chih Sun, La Jolla, CA (US); Divya Subramonian, La Jolla, CA (US)

(21) Appl. No.: 17/632,202

(22) PCT Filed: Aug. 27, 2020
(86) PCT No.: PCT/US2020/048264

§ 371 Date: Feb. 1, 2022

Related U.S. Application Data
(60) Provisional application No. 62/894,553, filed on Aug. 30, 2019.

(51) Int. Cl. G01N 33/72; G01N 33/58

(52) U.S. Cl. CPC G01N 33/72; G01N 33/582

(57) ABSTRACT

Identification and use of proteins fluorescently labeled and that undergo a change in fluorescence index upon binding bilirubin are described. Probes are disclosed which are labeled at a cysteine or lysine residue and also probes labeled at both cysteine and lysine with two different fluorophores. These probes are useful for determination of unbound bilirubin levels in a fluid sample.

### CROSS REFERENCE TO RELATED APPLICATION

This application claims the benefit of U.S. Provisional Application No. 62/894,553, filed Aug. 30, 2019, which is hereby expressly incorporated by reference in its entirety.

### STATEMENT REGARDING FEDERALLY SPONSORED R&D

This work was supported in part by SBIR Grant No. R44HD080412 from the National Institutes of Health. Consequently, the U.S. government may have certain rights to this invention.

### REFERENCE TO SEQUENCE LISTING

This application is filed with an electronic sequence listing entitled FFASC077WOSEQLIST.TXT, created on Aug. 24, 2020 which is 40 KB in size. The information in the electronic sequence listing is hereby expressly incorporated by reference in its entirety.

### FIELD

The present disclosure relates to the measurement of unbound bilirubin.

### BACKGROUND

Bilirubin is a product of hemoglobin turnover that is poorly soluble in water and is therefore largely associated with albumin in plasma. A small fraction of the total plasma bilirubin however is soluble in the aqueous phase. This unbound or free fraction is able to permeate the blood brain barrier and, at elevated levels, is neurotoxic [Ahlfors C E, Wennberg R P, Ostrow J D and Tiribelli C. Unbound (free) bilirubin: improving the paradigm for evaluating neonatal jaundice. Clin Chem 55: 1288-1299, 2009]. Under normal conditions, total serum bilirubin is maintained at low levels by a regulated balance between production and excretion of bilirubin. However, in newborns the mechanisms of regulation may not be sufficiently matured so that the production-excretion balance often favors accumulation, giving rise to the yellow color of jaundice in about 80% of newborns [Maisels M J and McDonagh A F. Phototherapy for neonatal jaundice. N Engl J Med 358: 920-928, 2008; Bhutani V K, Stark A R, Lazzeroni L C, Poland R, Gourley G R, Kazmierczak S, et al. Predischarge screening for severe neonatal hyperbilirubinemia identifies infants who need phototherapy. J Pediatr 2013; 162:477-82]. In most cases this imbalance is benign or may in fact be beneficial and for most newborns resolves spontaneously [Wennberg R P, Ahlfors C E, Bhutani V K, Johnson L H and Shapiro S M. Toward understanding kernicterus: a challenge to improve the management of jaundiced newborns. Pediatrics 117: 474-485, 2006; Gopinathan V, Miller N J, Milner A D and Rice-Evans C A. Bilirubin and ascorbate antioxidant activity in neonatal plasma. FEBS Lett 349: 197-200, 1994]. Concentrations of unbound bilirubin can rise to levels that are neurotoxic, resulting in deficits ranging from reversible hearing defects to the more severe neurological sequelae of kernicterus that in rare instances include death [Ahlfors C E, Wennberg R P, Ostrow J D and Tiribelli C. Unbound (free) bilirubin: improving the paradigm for evaluating neonatal jaundice. Clin Chem 55: 1288-1299, 2009].

Early intervention using phototherapy or exchange transfusion can treat bilirubin mediated neurotoxicity in neonates [Maisels M J and McDonagh A F. Phototherapy for neonatal jaundice. N Engl J Med 358: 920-928, 2008; Morris B H et al. Aggressive vs. conservative phototherapy for infants with extremely low birth weight. N Engl J Med 359: 1885-1896, 2008; Kuzniewicz M W, Escobar G J and Newman T B Impact of universal bilirubin screening on severe hyperbilirubinemia and phototherapy use. Pediatrics 124: 1031-1039, 2009]. Guidelines for intervention depend principally on total bilirubin levels, with account taken for gestational age and risk factors [Bhutani V K, Johnson L and Sivieri E M. Predictive ability of a predischarge hour-specific serum bilirubin for subsequent significant hyperbilirubinemia in healthy term and near-term newborns. Pediatrics 103: 6-14, 1999]. However, fundamental biochemical and increasing clinical evidence predicts that unbound bilirubin rather than total bilirubin should more accurately correlate with bilirubin mediated neurotoxicity [Ahlfors C E et al. Unbound (free) bilirubin: improving the paradigm for evaluating neonatal jaundice. Clin Chem 55: 1288-1299, 2009; Wennberg R P et al. Intervention guidelines for neonatal hyperbilirubinemia: an evidence-based quagmire. Curr Pharm Des 15: 2939-2945, 2009; Ahlfors C E et al. Unbound bilirubin predicts abnormal automated auditory brainstem response in a diverse newborn population. J Perinatol 29: 305- 309, 2009; Oh W et al. Influence of clinical status on the association between plasma total and unbound bilirubin and death or adverse neurodevelopmental outcomes in extremely low birth weight infants. Acta Paediatr 99: 673-678, 2010]. Therefore, unbound bilirubin should be superior to total bilirubin for identifying neonates at risk for bilirubin neurotoxicity [Ahlfors C E. Predicting bilirubin neurotoxicity in jaundiced newborns. Curr Opin Pediatr 22: 129-133, 2010; Watchko J F and Tiribelli C Bilirubin-Induced Neurologic Damage Mechanisms and Management Approaches. N Engl J Med 2013; 369:2021-30].

Aggressive phototherapy in premature infants is designed to maintain total bilirubin below 5 mg/dL [Morris B H et al. Aggressive vs. conservative phototherapy for infants with extremely low birth weight. N Engl J Med 359: 1885-1896, 2008]. Morris et al. found no difference in outcome (death and neurologic development impairment) for patients treated to maintain total bilirubin at less than 5 mg/dL and those maintained at less than 8 mg/dL. A follow up study found that outcomes were well correlated with unbound bilirubin but not total bilirubin [Oh W et al.]. Influence of clinical status on the association between plasma total and unbound bilirubin and death or adverse neurodevelopmental outcomes in extremely low birth weight infants. Acta Paediatr 99: 673-678, 2010]. This suggests that using total bilirubin for determining when to deliver phototherapy may have been misleading because the levels of total bilirubin were not coupled to unbound bilirubin, the toxic fraction of bilirubin. Decoupling of total bilirubin and unbound bilirubin may result from the presence of molecules that interfere significantly with bilirubin binding to albumin. For example, even if total bilirubin was as low as 1 mg/dL, displacement of just 0.2% of total bilirubin by interfering molecules would result in unbound bilirubin of 34 nM. This is an unbound bilirubin level that exceeds that thought to be toxic for term newborns and it is generally thought that much lower unbound bilirubin levels would be toxic for premature infants such as those in the Morris et al trial [Morris B H et al. Aggressive vs. conservative phototherapy for infants with extremely low birth weight. N Engl J Med 359: 1885-1896, 2008].

Several therapeutic and physiologic processes can produce molecular variants of bilirubin. The natural form of bilirubin is the Z,Z isomer (Z,Z-Bilirubin IXα). Upon exposure to light between 400 and 600 nm 3 variants are produced; the photoisomers Z,E-Bilirubin IXα and E,Z-Bilirubin IXα and the derivative Z-Lumirubin IXα. [Jana Jasproval et al. PLoS ONE DOI:10.1371, 2016; J. Jašprová et al. Neuro-inflammatory effects of photo-degradative products of bilirubin. Scientific Reports (2018) 8:7444]. Currently, the only FDA approved method for measuring unbound bilirubin levels in blood serum samples is the Arrows UB Analyzer which is based upon measuring the rate of bilirubin oxidation by HRP peroxidase. [H. Nakamura & Y, Lee, Micro-determination of unbound bilirubin in icteric newborn sera: an enzymatic method employing peroxidase and glucose oxidase. Clinica Chimica Acta, 79: 411- 417, 1977]. The bilirubin photoproducts may corrupt the peroxidase assay for unbound bilirubin because the assay assumes the decrease in bilirubin absorbance is due to the Z,Z isomer only. The absorption spectra of the Z,E and E,Z isomers overlaps with the Z,Z absorbance. [Antony F. McDonagh et al. Photoisomers: Obfuscating Factors in Clinical Peroxidase Measurements of Unbound Bilirubin? Pediatrics 2009; 123;67-76]. Because these and other photoisomers are much more soluble but much less toxic than the Z,Z isomer the peroxidase assay will report falsely elevated Bf levels. In addition to the bilirubin photoproducts, liver conjugates bilirubin with glucuronic acid for excretion by dissolution in bile where it eventually passes to stools. Depending upon the health status of the liver, some of the conjugated bilirubin may leak out of the liver into the blood circulation. Because the glucuronide modification increases the solubility relative to unconjugated bilirubin the conjugated bilirubin remains largely unbound and is often present at concentrations from 2 to 50 μM, with reference ranges of 0-2 μM. [Sanjiv Harpavat et al. An Infant with Persistent Jaundice and a Normal Newborn Direct Bilirubin Measurement. Clinical Chemistry 61:2 330-334 (2015)]. These μM concentrations may interfere with measurements of the nM (0-100 nM) unbound Z,Z bilirubin concentrations, especially with the peroxidase assay, which does not readily distinguish conjugated and unconjugated bilirubin.

Many drugs and metabolites can bind to albumin resulting in bilirubin displaced from its bound state on albumin and thereby increasing the unbound concentration of bilirubin whether or not the total bilirubin concentration increases [Spear M L et al. The effect of 15-hour fat infusions of varying dosage on bilirubin binding to albumin. JPEN J Parenter Enteral Nutr 9: 144-147, 1985; Amin S B. Effect of free fatty acids on bilirubin- albumin binding affinity and unbound bilirubin in premature infants. JPEN J Parenter Enteral Nutr 34: 414-420, 2010]. Especially important bilirubin displacing metabolites are free fatty acids (FFA). FFA are always present but are maintained at low levels and do not have a significant effect on healthy term newborns. However, under conditions of stress, for example due to sepsis, FFA levels can increases significantly [Nogueira A C et al. Changes in plasma free fatty acid levels in septic patients are associated with cardiac damage and reduction in heart rate variability. Shock 29: 342-348, 2008]. In addition to disease and stress, preterm infants in the NICU can produce extremely large increases in FFA levels due to parenteral nutrition with oil emulsions such as Intralipid® [Spear M et al. The effect of 15-hour fat infusions of varying dosage on bilirubin binding to albumin. JPEN J Parenter Enteral Nutr 9: 144-147, 1985; Amin S B. Effect of free fatty acids on bilirubin-albumin binding affinity and unbound bilirubin in premature infants. JPEN J Parenter Enteral Nutr 34: 414-420, 2010]. FFA bind albumin with high affinities similar to those of bilirubin. Unlike bilirubin, FFA have multiple high affinity binding sites so that only when an appreciable fraction of the albumin binding sites are occupied by FFA does bilirubin displacement become significant [Spear M L et al. The effect of 15-hour fat infusions of varying dosage on bilirubin b3inding to albumin. JPEN J Parenter Enteral Nutr 9: 144-147, 1985; Amin S B. Effect of free fatty acids on bilirubin-albumin binding affinity and unbound bilirubin in premature infants. JPEN J Parenter Enteral Nutr 34: 414-420, 2010]. Which newborns receiving Intralipid® will produce large enough quantities of FFA to displace bilirubin cannot be predicted easily because factors such as gestational age, enzymatic activity, adiposity and others play a role. [Spear M L et al. The effect of 15-hour fat infusions of varying dosage on bilirubin binding to albumin. JPEN J Parenter Enteral Nutr 9: 144-147, 1985; Amin SB. Effect of free fatty acids on bilirubin-albumin binding affinity and unbound bilirubin in premature infants. JPEN J Parenter Enteral Nutr 34: 414-420, 2010]. Monitoring the unbound concentration of FFA (FFAu) during lipid infusion reveals that elevated FFAu levels greatly increase unbound bilirubin to dangerous levels [Hegyi T et al. Unbound Free Fatty Acids from Preterm Infants Treated with Intralipid Decouples Unbound from Total Bilirubin Potentially Making Phototherapy Ineffective Neonatology 2013; 104:184-187; Hegyi et al. Effects of Soybean Lipid Infusion on Unbound Free Fatty Acids and Unbound Bilirubin in Preterm Infants J Pediatr 2017; 184:45-50]. In addition, because the unbound levels of these metabolites are dependent upon many patient-specific factors, only by directly monitoring unbound bilirubin during Intralipid® infusion can those infants at risk for bilirubin neurotoxicity be identified. This is particularly true for bilirubin because the elevated plasma levels of FFA caused by increasing Intralipid® concentrations produce elevated unbound bilirubin concentrations without changing the total bilirubin concentration.

Intracellular lipid binding proteins (iLBP) are a family of low-molecular weight single chain polypeptides. There are four recognized subfamilies. Subfamily I contains proteins specific for vitamin A derivatives such as retinoic acid and retinol. Subfamily II contains proteins with specificities for bile acids, eicosanoids, and heme. Subfamily III contains intestinal type fatty acid binding proteins (FABPs). Subfamily IV contains all other types of fatty acid binding protein [Haunerland N H and Spener F. Fatty acid-binding proteins, insights from genetic manipulations. Prog Lipid Res 43: 328-349, 2004] including an FABP that binds bilirubin with low affinity [Di Pietro S M and Santome J A. Isolation, characterization and binding properties of two rat liver fatty acid-binding protein isoforms. Biochim Biophys Acta 1478: 186-200, 2000]. The entire family is characterized by a common 3-dimensional fold. Ligand binding properties of the different subfamilies overlap considerably. The wild type proteins of subfamily I [Richieri G V et al. Fatty acid binding proteins from different tissues show distinct patterns of fatty acid interactions. Biochemistry 39: 7197-7204, 2000] and subfamily II both bind fatty acids as well as their native ligands. Moreover, single amino acid substitutions are able to interconvert the ligand binding properties of proteins of subfamilies I and II [Jakoby M G et al. Ligand-protein electrostatic interactions govern the specificity of retinol- and fatty acid-binding proteins. Biochemistry 32: 872-878, 1993]. The disclosure of each reference set forth herein is expressly incorporated by reference in its entirety, and for the disclosure referenced herein.

### SUMMARY

Described herein are compositions, kits, devices, systems, and methods related to near infrared (NIR) Bf sensors that have improved fluorophores, lower levels of interferences from drugs and metabolites than other methods, a disposable cartridge that enables Bf determination in a single step with less than 5 μL of undiluted blood samples, and methods for calibrations of the assay. As described herein probes can comprise iLBPs labeled with a fluorophore. Probes of unbound bilirubin (Bf or UB) are disclosed that undergo a change in fluorescence index upon binding bilirubin and which probes may be used to measure levels of unbound bilirubin in fluids. The fluorescence index may be, for example, wavelength, intensity, polarization, lifetime, or any measurable quantity of the fluorescence. The unbound bilirubin probes disclosed herein do not significantly bind or undergo a significant fluorescent change in the presence of other analytes present in fluids in which unbound bilirubin levels are determined. The probes of unbound bilirubin described herein can be used in the diagnosis and treatment of hyperbilirubinemia and to assess the risk of bilirubin toxicity. Non-responder probes are also identified that do not bind bilirubin and do not undergo a change in fluorescence index in the presence of bilirubin and whose fluorescence is not affected by other analytes generally present in fluids in which unbound bilirubin levels are determined. Together, an unbound bilirubin probe with a first fluorophore and a non- responder probe with a different fluorophore can produce a Bf sensor, which in the presence of Bf undergoes a change in the ratio of fluorescence indexes from the first fluorophore relative to the second fluorophore. Together with the cartridge containing Bf sensor and a dedicated fluorescence reader the assay is designated the UBCheck assay.

Some embodiments provided herein relate to sensors for measuring free bilirubin in a sample. In some embodiments, the sensors include a bilirubin responsive probe labeled with a first fluorophore; and a non-responder probe labeled with a second fluorophore. In some embodiments, the first and second fluorophores excite at the same wavelength, and wherein the first and second fluorophore emit a fluorescence at different wavelengths. In some embodiments, the bilirubin responsive probe includes a first intracellular lipid binding protein (iLBP), wherein the first iLBP has a peptide sequence comprising SEQ ID NO: 1, and includes arginine substituting for fourteen accessible lysines (KR14, as set forth in SEQ ID NO: 2); a C terminal double His tag linker (C2XH11) having a sequence as set forth in SEQ ID NO: 3; an N terminal addition of MGI; and no more than 62 amino acid substitutions and additions including a single cysteine. In some embodiments, the bilirubin responsive probe comprises a sequence of any one of the probes as set forth in Table 1. In some embodiments, the non-responder probe includes a second iLBP, wherein the second iLBP has a peptide sequence comprising SEQ ID NO: 1, and includes substitutions at positions 72, 73, 74, 126, and 131; a substitution to Cys at any one of positions 27, 31, 33, 54, 73, 74, 76, or 98; no more than three additional amino acid substitutions; and a C terminal double His tag linker (C2XH11) having a sequence as set forth in SEQ ID NO: 3. In some embodiments, the non-responder probe comprises a sequence of any one of the probes as set forth in Table 2. In some embodiments, the first fluorophore and the second fluorophore are different fluorophores. In some embodiments, the bilirubin responsive probe comprises a single cysteine to which the first fluorophore is attached. In some embodiments, the non-responder probe comprises a single cysteine to which the second fluorophore is attached. In some embodiments, the first fluorophore and the second fluorophore are excited at the same or about the same wavelengths. In some embodiments, the first fluorophore is LICOR 700DX maleimide or LICOR 800CW maleimide attached to a cysteine substitution. In some embodiments, the bilirubin responsive probe is configured to bind to an unconjugated IX-α (Z,Z) isomer of bilirubin. In some embodiments, the bilirubin responsive probe is configured to minimally bind to conjugated bilirubin (below 4 mg/dl). In some embodiments, the bilirubin responsive probe is configured to not bind to Z,E or E,Z photoisomers of bilirubin, to lumirubin, to fatty acids, to any other naturally occurring blood components, and/or to neonatal drugs. In some embodiments, the neonatal drugs is not spironolactone. In some embodiments, the non-responder probe is configured to not bind to unconjugated IX-α (Z,Z) isomer of bilirubin or conjugated bilirubin. In some embodiments, the non-responder probe is configured to not bind to Z,E or E,Z photoisomers of bilirubin, to lumirubin, to fatty acids, to any other naturally occurring blood components, and/or to neonatal drugs. In some embodiments, the first fluorophore is LICOR 700DX maleimide, the second fluorophore is LICOR 800CW maleimide attached to a cysteine substitution, and when the first fluorophore is LICOR 800CW maleimide, the second fluorophore is LICOR 700DX maleimide. In some embodiments, the first fluorophore or the second fluorophore is attached to a cysteine substitution and wherein the cysteine substitution is at position 22, 24, 25, 26, 27, 29, 30, 33, 54, 74, 76, 97, or 98 of SEQ ID NO: 1. In some embodiments, an emission intensity of the first fluorophore or the second fluorophore is not affected by the absorbance of blood components selected from bilirubin and hemoglobin. In some embodiments, the bilirubin responsive probe or the non-responder probe further comprising at least one linker.

Some embodiments provided herein relate to compositions that include any of the sensors described herein. In some embodiments, the compositions include a free bilirubin (Bf) sensor. In some embodiments, the sensor includes a first intracellular lipid binding protein (iLBP) that binds bilirubin and is labeled with a first fluorophore; and a second iLBP that does not bind bilirubin and is labeled with a second fluorophore, wherein the second fluorophore is not attached to the first iLBP, wherein the first fluorophore and the second fluorophore excite at the same wavelength, wherein the emission wavelength of the first fluorophore and the second fluorophore are different, and wherein the second fluorophore does not change its emission in the presence of bilirubin. In some embodiments, the first fluorophore is LICOR 700DX maleimide and the second fluorophore is LICOR 800CW maleimide, or wherein the first fluorophore is LICOR 800CW maleimide and the second fluorophore is LICOR 700DX maleimide. In some embodiments, a change in ratio of fluorescence index is measured at two different wavelengths and used to determine a concentration of unbound bilirubin. In some embodiments, an emission intensity of the first fluorophore or the second fluorophore is not affected by the absorbance of blood components selected from bilirubin and hemoglobin.

Some embodiments provided herein relate to solid substrates that include the sensors as described herein or that include the compositions described herein. In some embodiments, the bilirubin responsive probe and/or the non-responder probe is attached to the solid substrate. In some embodiments, the solid substrate is a Ni-polystyrene, Ni-latex, or Ni-agarose bead. In some embodiments, the Ni-polystyrene, Ni-latex, or Ni-agarose bead comprises iron. In some embodiments, the bilirubin responsive probe or the non-responder probe comprises substitutions 7R 16R 20R 29R 37R 46R 50R 88R 92R 94R 100R 125R 129R and/or 130R (KR14) as set forth in SEQ ID NO: 3. In some embodiments, the bilirubin responsive probe and/or the non-responder probe comprises a tag and the solid substrate comprises a receptor for the tag. In some embodiments, the tag comprises one or more of His-tag, biotin, Flag-epitope, c-myc epitope, HA-tag, glutathione-S-transferase (GST), maltose binding protein (MBP), a chitin binding domain (CBD), Thioredoxin, β-Galactosidase, VSV-Glycoprotein, calmodulin binding protein, a polystyrene (PS) hydrophobic tag, or a metal affinity tag. In some embodiments, the tag is a poly-histidine tag and the solid substrate comprises an immobilized metal chelate. In some embodiments, the first fluorophore is attached to a cysteine residue on the bilirubin response probe. In some embodiments, the second fluorophore is attached to a cysteine residue on the non-responder probe.

Some embodiments provided herein relate to methods of calibrating bilirubin sensors to determine Kd and Rm. In some embodiments, the methods include mixing the sensor of any one of the aforementioned sensors with an aqueous sample of a known concentration of bilirubin, Bt, measuring a fluorescence, and determining calibration parameters from the measured fluorescence by fitting with equation (1):

\overline{R=(\sqrt{PT²+2PT(K_{d}−BT)+K_{d}²+2BTK_{d}}+BT²})((RₘRₒ²−Rₘ²Rₒ)r²+(Rₘ²−Rₒ²)r+Rₒ−Rₘ)+((PT+K_{d}+BT)RₘRₒ²+(PT−K_{d}−BT)Rₘ²Rₒr²+(((−PT−K_{d}+BT)Rₒ²+((−2PT)+2K_{d}−2BT)RₘRₒ+((−PT)−K_{d}+BT)Rₘ²r+(PT−K_{d}−BT)Rₒ+(PT+K_{d}+BT)Rₘ)/((2BTRₒ²+2(PT+K_{d}+BT)RₘRₒ−2K_{d}Rₘ²)r²+((−2(PT+K_{d}+BT)Rₒ+((−2PT+2(K_{d}+BT))Rₘ*r+2PT)

wherein R is a measured fluorescence ratio ((I_{λ1}/I_{λ2}), I_{λ1} is a fluorescence intensity from a first fluorophore at wavelength λ1 with background from the sample subtracted, I_{λ2} is a fluorescence intensity from a second fluorophore at wavelength λ2 with background from the sample subtracted, Rₒ is a ratio in the absence of bilirubin, BT is a total bilirubin concentration, PT is a responder probe concentration, r is a I_{λ2}/I_{λ1} ratio of the bilirubin probe fluorophore in the absence of the second fluorophore, K_{d} is an equilibrium dissociation constant of the bilirubin probe, and Rm is a ratio R extrapolated to infinite BT.

Some embodiments provided herein relate to methods of measuring a concentration of free bilirubin [B_{f}] in a sample. In some embodiments, the methods include measuring a baseline fluorescence of the sample; applying a sample to any one of the sensors as described herein; measuring a sample fluorescence; subtracting the baseline fluorescence from the sample fluorescence to obtain a measured fluorescence; and determining the concentration of [B_{f}] from the measured fluorescence. In some embodiments, the steps of measuring a baseline and/or subtracting a baseline are optionally performed. In some embodiments, equation (1) is used to calibrate the sensor and equation (2)

$$[ {B}_{f} ] = {K}_{d} \frac{( rRm - 1 )}{( {rR}_{o} - 1 )} \frac{( R - {R}_{o} )}{( {R}_{m} - R )} \qquad ( 2 )$$

is used to determine [B_{f}], wherein R is a measured fluorescence ratio ((I_{λ1}/I_{λ2}), I_{λ1} is a fluorescence intensity from a first fluorophore at wavelength λ1, I_{λ2} is a fluorescence intensity from a second fluorophore at wavelength λ2, Rₒ is a ratio in the absence of bilirubin, r is a I_{λ2}/I_{λ1} ratio of the probe in the absence of the second fluorophore, Kd is a dissociation constant, Rm is a minimum R value at 00 Bf , and Rm is a R at bilirubin saturation of the probe.

In some embodiments, the sample is mixed with one or more carrier macromolecules for the bilirubin. In some embodiments, the one or more carrier macromolecules comprise albumin, lipid binding proteins, lipid vesicles, or cyclodextrin. In some embodiments, the sensor is attached to a solid support. In some embodiments, the Bf concentrations are determined using disposable microfluidics devices, which optionally allow measurements of undiluted blood samples. In some embodiments, the sample is from a human, an animal, or a plant. In some embodiments, the sample is whole blood, blood plasma, blood serum, urine, CSF, saliva, gastric juices, interstitial fluid, or lymph. In some embodiments, the sample is from patients receiving intravenous infusion of oil emulsions. In some embodiments, the sample is from patients that are receiving drugs that displace bilirubin from albumin and/or such patients may be producing, from the infused oil emulsion, disease or stress, molecules that displace bilirubin from albumin. In some embodiments, the sample is from patients that are undergoing phototherapy, transfusion or other therapies that reduce bilirubin levels. In some embodiments, Ro is obtained by photobleaching the sample, thereby obtaining a zero level measurement.

Some embodiments provided herein relate to cartridges. In some embodiments, the cartridges are configured to measure bilirubin in a sample. In some embodiments, the cartridges include a base, a lens configured to couple to the base, and comprising a sample port for receiving a sample, and a substrate having a bilirubin responsive probe, a non-responder probe, and anti-hemoglobin peptides immobilized thereto. In some embodiments, the base is treated with UV light at a wavelength ranging from about 145 nm to about 225 nm, thereby phototreating polystyrene polymer chains to link to polymer chains of the substrate. In some embodiments, the base is a polystyrene base. In some embodiments, the base comprises a material having a dark color configured to reduce reflection intensity of exciting light at 660 nm. In some embodiments, the lens is an acrylic lens. In some embodiments, the lens is treated with O₂ plasma. In some embodiments, coupling the lens to the polystyrene base forms a channel with a depth of about 0.1 mm or less, and seals the cartridge. In some embodiments, the sample is whole blood, blood plasma, blood serum, urine, CSF, saliva, gastric juices, interstitial fluid, or lymph. In some embodiments, the sample is an undiluted sample. In some embodiments, the cartridge is configured to measure bilirubin at equilibrium. In some embodiments, the cartridge is calibrated by traceability of a bilirubin standard. In some embodiments, the bilirubin standard is commercially available bilirubin that is used to calibrate a probe, wherein the probe is used to calibrate a calibration complex, and wherein the calibration complex is used to calibrate the cartridge.

Some embodiments provided herein relate to kits. In some embodiments, the kits include one or more collection devices for collecting a sample from a patient, any one of the sensors as described herein or any one of the compositions as described herein including one or more probes in a suitable carrier, and one or more reference standards comprising a known concentration of unbound bilirubin below and/or above medical decision levels. In some embodiments, the one or more reference standards are optional.

### BRIEF DESCRIPTION OF THE DRAWINGS

FIG. 1 depicts an embodiment of emission spectra of a bilirubin sensitive probe labeled with LICOR 700DX maleimide and a non-responder probe labeled with LICOR 800CW maleimide mixed in a fluorimeter showing intensities at zero BT and depicting a titration of mixing with increasing Bf which shows quenching of emissions at 710 nm and no change in 805 nm and a corresponding decrease in the 710/805 ratio.

FIG. 2 depicts an embodiment of free probe calibration data with fits and resulting parameters. FIG. 2 depicts an embodiment of free probe, with a concentration of 1.2 nM, calibration data with the fit of equation 1 which yields the parameters, Kd, Rm and Qs=Rm/Ro.

FIG. 3 depicts an embodiment of calibration of a cartridge lot with calibrated bilirubin-human serum albumin (HSA) complexes that generate fixed Bf values. The measured R values at each Bf is fitted with equation 3 to yield Kd (in nM), Rm and the fit quality as determined by the weighted χ².

FIG. 4 depicts an embodiment of the effects of dilution on Bf levels in the presence of a strong displacer, oleic acid.

FIG. 5 depicts concentrations of unbound bilirubin (Bf) as a function of dilution of hemoglobin (Hb).

FIG. 6 depicts an embodiment of the effects of newborn intensive care unit (NICU) drugs that are potent displacers of bilirubin from albumin.

FIG. 7 depicts an embodiment of a Bf assay, showing lower sensitivity to conjugated bilirubin (cBR) than the Arrows peroxidase method.

FIGS. 8A and 8B depict an embodiment of increasing intralipid concentration which in the absence of lipolysis reduces Bf which partitions into the triglyceride (FIG. 8A) and while in the presence of heparin which activates lipolysis which generates unbound FFA that increases Bf by displacing bilirubin from albumin (FIG. 8B).

FIGS. 9A and 9B depict an embodiment of data showing that the Bf assay only detects the Z,Z isomer of bilirubin but Arrows is sensitive to Z,Z and all photoisomers.

FIGS. 10A-10D schematically represent multiple views of an embodiment of a polystyrene disposable sample cartridge base.

FIGS. 11A-11D schematically represent multiple view of an embodiment of a lens that is configured to couple to the polystyrene disposable sample cartridge base shown in FIGS. 10A-10D.

FIG. 12 depicts the disposable cartridge with the Bf sensor spot and sample port defined.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

U.S. Pat. Nos. 5,470,714, 6,444,432, 7,601,510, 9,134,317, 9,529,003, and 9,817,004 describe methods for generating probes for the determination of unbound analytes and are expressly incorporated herein by reference in their entireties and for such disclosure. These probes were constructed using either native or mutant forms of proteins from the intracellular lipid binding protein (iLBP) family. As discussed above, this family includes fatty acid binding proteins (FABPs) [Banaszak et al. Lipid-bindingproteins A family of fatty acid and retinoid transport proteins. Adv Protein Chem 45: 89-151, 1994; Bernlohr D A et al. Intracellular lipid-binding proteins and their genes. Annu Rev Nutr 17: 277- 303, 1997]. iLBPs are intracellular proteins of approximately 15 kDa molecular weight and have a binding site that in the wild type proteins binds 1 or 2 FFA as well as other metabolites.

The patents and publications disclosed and described herein, each of which are expressly incorporated by reference herein in its entirety, and for any disclosure specifically referenced herein, describe unbound bilirubin (UB), also referred to herein as free bilirubin (Bf) ratio sensors composed of bilirubin sensitive iLBPs with one fluorophore and a second fluorophore free or attached to protein that does not bind or respond to bilirubin. The present disclosure describes improvements to the prior patents and publications, including, for example near infrared (NIR) Bf sensors that have improved fluorophores, lower levels of interferences from drugs and metabolites than other methods, a disposable cartridge that enables Bf determination in a single step with less than 5 μL of undiluted blood samples, and methods for calibrations of the assay.

The bilirubin sensitive NIR fluorescently labeled iLBP mutants described herein are developed with improved bilirubin specificity and sensitivity. Prior disclosures, for example, U.S. Pat. No. 9,529,003 describes iLBPs labeled with fluorophores primarily at lysine, terminal amino groups, or cysteine. As disclosed in U.S. Pat. No. 9,134,317, labeling at a single cysteine improves sensitivity and specificity for detection of ligand binding to iLBPs by eliminating fluorophores labeling at multiple sites (wild type FABP (including the protein having a sequence as set forth in SEQ ID NO: 1) or mutated FABP proteins (such as a mutated protein having the sequence as set forth in Tables 1 and 2 do not have a cysteine residue)). Because fluorophores at most sites do not change their fluorescence upon ligand binding, labeling at most sites reduces the fluorescence signal to noise ratio upon ligand binding. The iLBPs have as many as 14 surface accessible lysines in addition to the terminal amino group all of which may be labeled with an amino reactive fluorophore. One improvement of the present disclosure is related to the use of newly available cysteine specific forms (maleimide) of the two fluorophores LICOR 700DX and LICOR 800CW. The LICOR 700DX maleimide was not available or known previous to the time of the prior disclosures and LICOR 800CW maleimide was not investigated in U.S. Pat. No. 9,529,003. The bilirubin sensitive iLBPs disclosed herein have specific single cysteine mutations that are labeled with LICOR 700DX-maleimide. Also developed are iLBP mutants that do not exhibit a change in fluorescence in the presence of bilirubin and are labeled at a single cysteine mutation with LICOR 800CW-maleimide.

The probes disclosed herein may be insensitive to more than 50 of the drugs most prescribed to neonates (e.g., those listed in Table 4), to conjugated bilirubin, to photo isomers of bilirubin, to intravenous lipid emulsions used to provide parenteral nutrition to premature or otherwise at-risk infants, and are insensitive to free fatty acids.

The present disclosure also relates to devices for determination of unbound bilirubin concentrations in a sample. In some embodiments, the devices include a disposable plastic cartridge that contains the NIR fluorescent Bf sensor as a dried spot at the center of a blood sample micro-channel (e.g., having a volume of 1-50 μL, in some embodiments 5 μL). Fluorescence from the cartridge may be measured after applying the blood sample, by inserting the cartridge into a fluorescence reader developed specifically for this measurement. This configuration of disposable sample cartridge and reader may allow blood levels of unbound bilirubin to be measured in a single step, and in some embodiments using microliter volumes of samples.

Drying the Bf sensor on the cartridge results in bilirubin mediated incomplete quenching of the fluorescence of the bilirubin sensitive probe. This requires a new method of analysis for characterization of the sensor and for calculation of the unbound bilirubin concentration from the ratio of fluorescence from the bilirubin sensitive probe divided by the fluorescence from the bilirubin insensitive probe.

Accordingly, some embodiments provided herein are directed to methods for identification of fluorescently labeled proteins (or iLBP muteins) that are highly specific for unbound bilirubin and fluorescently labeled iLBP muteins that are non-responsive to bilirubin. Some embodiments are directed to the use of different near infrared (NIR) fluorophores on the bilirubin responsive iLBP muteins and on the non-responder iLBP mutein. When combined in a Bf sensor the presence of Bf will produce a change in the ratio of the index of the two different NIR- fluorophores. Embodiments provided herein relate to methods for using the two fluorescently labeled proteins to produce a fluorescent ratio sensor for determining unbound bilirubin concentrations in samples ranging from simple aqueous solutions to complex biological samples including human fluids (blood, CSF, urine, interstitial fluid). In some embodiments, the methods include generating the probes using the methods of U.S. Pat. Nos. 7,601,510 and 9,529,003 and generating a ratio Bf sensor by use of a second fluorescently labeled non-responder protein. One or both fluorescent proteins may be free in solution or one or both may be attached to a solid substrate (matrix) or resin polymer, such as polydextran, or polystyrene, for example. In some embodiments, the methods include calibrating the sensors to determine the calibration constants of the sensor using equation (1). Some embodiments are related to linking probes to solid substrates. The characteristics of the probes linked to solid substrates may be examined to assess accurate and precise determination of unbound bilirubin levels in such devices. Such characteristics may include, for example, effects of probe dissociation, albumin buffering, or bilirubin binding to polymer but not probe and equilibrium rates on the polymer. In some embodiments, the Bf sensor specificity may be refined by testing against a panel of potential interferants, including common metabolites, drugs, bilirubin photoisomers, conjugated bilirubin, or other analyte contributions equivalent to less than 1 nM unbound bilirubin or some medically suitable level. In some embodiments, the methods further include testing quantitation by measurement of unbound bilirubin in defined human plasma, serum or whole blood spiked with bilirubin to ensure specificity for unbound bilirubin in human blood samples. In some embodiments, the methods further include calculating unbound bilirubin concentrations as described in equation (2).

Some embodiments are directed to probes based upon iLBPs, such as the lipid binding protein, which corresponds to SEQ ID NO: 1, which includes one or more amino acid substitutions and a fluorophore (see examples in Tables 1 and 2). In some embodiments, the fluorophore is attached to a cysteine residue of the iLBPs having only a single reactive cysteine. In some embodiments, the bilirubin sensitive iLBP binds to the IXα-Z,Z isomer of bilirubin, but does not significantly bind to the photoisomers Z,E, E,Z and lumirubin, to conjugated bilirubin, nor to fatty acids and the non-responder iLBP also has only a single reactive cysteine and does not bind or respond to any of these analytes.

In some embodiments, the bilirubin sensitive probe corresponds to the lipid binding protein of SEQ ID NO: 1 with an N terminal MGI substitution, a C terminal double HIS tag substitution C2XH11 (SEQ ID NO: 3) and one or more amino acid substitutions at positions selected from 14, 18, 23, 28, 24, 25, 26, 27, 29, 30, 33, 38, 54, 60, 73, 74, 76, 97, 98, 106, 115, 117, or 132 of SEQ ID NO: 1.

In some embodiments, the fluorophore for the bilirubin responsive probe is attached to a cysteine substitution at positions 22, 24, 25, 26, 27, 29, 30, 33, 54, 73, 74, 76, 97 or 98 of SEQ ID NO: 1.

In some embodiments, the probe is substituted with arginine at positions 7R 16R 20R 29R 37R 46R 50R 88R 92R 94R 100R 125R 129R and/or 130R (KR14—SEQ ID NO: 2) of SEQ ID NO: 1, except when the position 29 is mutated to cysteine.

In some embodiments, the probe includes a linker (C2XH11) at the C- terminus of SEQ ID NO: 1 with sequence RGAASHHHHHHSHRATPNTSPHHHHHHH (SEQ ID NO: 3).

In some embodiments, a polynucleotide template encodes iLBP muteins having a cleavable or non-cleavable affinity tag. In some embodiments, the template polynucleotide template encodes iLBP muteins having poly-histidine affinity tags and the solid substrate includes an immobilized metal chelates.

In some embodiments, the bilirubin responsive and non-responder iLBP muteins are labeled with a single fluorophore at a pH of less than 8. At a pH of less than 8, the fluorophore may react with the cysteine sidechains. In some embodiments, the fluorophores are thiol specific fluorophores that excite at a wavelength of about 660 nm and emit at a wavelength of about 700 nm and 819 nm, such as a LICOR 700DX-maleimide and LICOR 800CW maleimide.

In some embodiments, a second fluorophore is provided by addition of a fluorophore to a non-responder probe, such as a non-responder iLBP mutein. The non- responder iLBP is also labeled with a single fluorophore that preferentially reacts with the cysteine sidechain at a pH of less than 8. In some embodiments, the fluorophore is LICOR 800CW-maleimide or Biotium CF800 Maleimide. In some embodiments, the non-responder iLBP has zero or a significantly reduced response in its fluorescence index upon exposure to bilirubin, compared to the fluorescence of the bilirubin responsive (bilirubin binding) iLBP mutein probe.

In some embodiments, the non-responder probes are based upon iLBPs, such as the lipid binding protein which corresponds to SEQ ID NO: 1, which includes one or more amino acid substitutions and a fluorophore (see examples in Table 2). In some embodiments, the fluorophore is attached to a cysteine residue, of the iLBPs having only a single reactive cysteine.

In some embodiments, the non-responder probe corresponds to the lipid binding protein of SEQ ID NO: 1 having one or more amino acid substitutions at positions selected from 14, 18, 20, 23, 27, 29, 33, 54, 72, 73, 74, 76, 98, 100, 117, 126, or 131.

In some embodiments, the fluorophore for the non-responder probe is attached to a cysteine substitution at positions 27, 31, 33, 54, 73, 74, 76, or 98 of SEQ ID NO: 1.

Some embodiments provided herein are directed to compositions having an iLBP mutein labeled with a first fluorophore and a second fluorophore that is attached to a separate unattached iLBP which does not bind bilirubin. In some embodiments, the first fluorophore and the second fluorophore are capable of excitation at the same wavelength and the emission wavelength of the first fluorophore and the second fluorophore are different. In some embodiments, the second fluorophore is not affected (does not change its emission) in response to bilirubin binding to the bilirubin non-responder iLBP mutein and/or the non- responder iLBP does not bind bilirubin. In some embodiments, the first fluorophore is LICOR 700DX maleimide and the second fluorophore is LICOR 800CW maleimide.

In some embodiments, a change in ratio of fluorescence index is measured at two different wavelengths and this ratio is used to determine the unbound bilirubin concentration.

In some embodiments, the index is the emission intensities of the fluorophore(s) attached to the iLBP muteins, which as described herein are not affected significantly by light absorbance (above 600 nm) of blood components such as bilirubin and hemoglobin. In some embodiments, the systems and methods described herein are independent of hemolysis. Hemolysis causes significant interference due to hemoglobin and heme in the sample. However, the methods, systems, and compositions provided herein overcome the interference associated with hemolysis. In some embodiments a peptide is added to the solid substrate on which the Bf probes are attached, and the peptide eliminates or significantly reduces interference from hemolysis.

In some embodiments, the first fluorophore is attached to a cysteine and is LICOR 700DX-maleimide and the second fluorophore is also attached to a cysteine on a different iLBP and is LICOR 800CW-maleimide.

In other embodiments the first fluorophore is LICOR 800CW-maleimide and is attached to the bilirubin sensitive mutein and the second fluorophore is LICOR 700DX- maleimide and is attached to the non-responder mutein.

In other embodiments described herein, a second different fluorophore is attached to a protein that does not bind bilirubin. A probe combining the first fluorophore attached to the bilirubin responsive iLBP mutein and a probe combining the second fluorophore is attached to a bilirubin non-responder iLBP mutein, and therefore a mixture of the responsive and non-responder probes yields a Bf sensor that changes its ratio of a fluorescence index measured at two different wavelengths in response to bilirubin. The second fluorophore can have a longer or shorter emission wavelength than the first (responsive iLBP mutein) fluorophore, but both fluorophores should have a common excitation wavelength. For example, in some embodiments the first (protein linked) fluorophore is LICOR 700DX-maleimide and examples of the second include but are not limited to LICOR 800CW-maleimide, and or Biotium CF800 maleimide linked to a bilirubin non-responder protein. One or both fluorescently labeled proteins may be free in solution or embedded in another polymer or solid substrate. This arrangement has the desirable benefit that the concentrations of the fluorophores can be adjusted so that emission intensities of both fluorophores are similar even when the maximum excitation wavelength for the second fluorophore is different than for the first fluorophore. This type of ratio probe using a second different fluorophore, not attached to the same protein as the first fluorophore, eliminates the problem of energy transfer quenching of one of the two fluorophores by the other, typically when both fluorophores are located on the same protein.

In some embodiments, the second fluorophore is attached to an acceptor protein. In some embodiments, the probe includes substitutions 7R 16R 20R 29R 37R 46R 50R 88R 92R 94R 100R 125R 129R and 130R (KR14—SEQ ID NO: 2) of SEQ ID NO: 1, except when one of these positions has a cysteine substitution.

In some embodiments, the probe includes a C terminal linker C2XH11 composed of the sequence RGAASHHHHHHSHRATPNTSPHHHHHHH (SEQ ID NO: 3).

Embodiments provided herein are directed to probes in which the fluorophore is attached to a cysteine residue, such as LICOR 700DX maleimide, LICOR 800CW maleimide, LICOR, IRDye 680LT maleimide, Alexafluor 680 maleimide or Biotium CF800 Maleimide.

In some embodiments, any of the probes as described above may include two or more tags at the C or N-terminus of the probe in combination with one or more linkers for attachment to a solid support.

In some embodiments, the probe is attached to a solid support using two His-tags and two linkers.

Embodiments provided herein are directed to compositions which contain probes as described above.

Some embodiments provided herein relate to a bilirubin sensitive probe (e.g. LICOR 700DX-maleimide) and a bilirubin non-responder probe (e.g. LICOR 800CW-maleimide) free in solution. In some embodiments, the bilirubin sensitive probe and the bilirubin non-responder probe are attached to solid substrates in solution.

Embodiments provided herein are directed to a solid substrate thatincludes any of the probes described above attached to the solid substrate. In some embodiments, the solid substrate is a polystyrene or latex bead, Ni-polystyrene bead, optionally containing iron. The probes selected for attachment to the solid substrate may contain any of the modifications described above alone or in combination including, but not limited to N-terminal modifications and C-terminal modifications, linkers and substitutions of surface lysines (KR14). The two probes with the different emission wavelengths may be immobilized on the same or different solid substrates that are nanoparticles or beads.

In some embodiments, the probe is tagged for attachment to the solid substrate. In some embodiments, the tag includes one or more of His-tag, biotin, Flag-epitope, c-myc epitope, HA-tag, glutathione-S-transferase (GST), maltose binding protein (MBP), a chitin binding domain (CBD), Thioredoxin, β-Galactosidase, VSV-Glycoprotein, calmodulin binding protein, a polystyrene (PS) hydrophobic tag, or a metal affinity tag.

Some embodiments are directed to a solid substrate in which the probe has a tag and the solid substrate includes a receptor for the tag. In some embodiments, the tags are poly-histidine tags with or without additional linkers and the solid substrate includes immobilized metal chelates.

Other embodiments include bilirubin probes that are attached to solid substrates that are nanoparticles, including but are not limited to polymers such as dextran, polystyrene, latex, agarose beads or Ni-NTA polystyrene beads, optionally containing iron. These nanoparticle substrates can be further immobilized on a macroscopic surface. Examples of the use of such surfaces include but are not limited to, channels of disposable microfluidics devices including single use sample cartridges. Examples of bilirubin probes that are designed to be immobilized on surfaces include, but are not limited, to combinations of one each from Tables 1 and 2 whose double His tags and linkers allow them to chelate with various metal ligands on various polymer resins including but not limited to Ni, Co or Cu on polystyrene, latex or agarose beads. Such beads with attached probes can be used free in solution and the bilirubin responsive and non-responder probes can be added to the same or different beads and for both configurations the two different fluorophores are sufficiently well separated so as to eliminate energy transfer and thereby obtain a ratio response to bilirubin binding. In some embodiments, the substrate is a microfluidic device or multi-well plate. In some embodiments, the substrates are included in the detection devices described herein, such that the substrate is attached to a surface of the cartridge.

Embodiments provided herein are directed to an iLBP mutein having a single cysteine labeled with a fluorescent dye. In some embodiments, any surface lysines or any other cysteines with fluorescent labeling activity under cysteine/lysine-specific labeling conditions are replaced with another amino acid, including, for example alanine or arginine. In some embodiments which utilize an iLBP mutein template corresponding to SEQ ID NO: 1, the lysine at position 27 is highly reactive and may be mutated, typically to alanine, unless the label is directed to that position.

In some embodiments, bilirubin is complexed with a carrier macromolecule such as albumin, lipid binding proteins, lipid vesicles or cyclodextrin. The complex of bilirubin and the carrier macromolecule buffers the concentration of the unbound bilirubin which provides clamping of a level of unbound bilirubin. In some embodiments, the carrier macromolecule is albumin. In further embodiments, the albumin is human serum albumin (HSA) which has a larger affinity for bilirubin than, for example, bovine serum albumin and therefore may be a more preferred albumin buffer for bilirubin in some embodiments.

Embodiments provided herein are directed to methods of calibrating the bilirubin probes by mixing in an aqueous media the sensor with samples of bilirubin of increasing total bilirubin concentrations (BT), measuring the ratio of fluorescence intensities R of the sensor at each concentration, and determining the calibration parameters (Kd, Rm, and Ro) from the measured fluorescence by fitting with the equation (1), wherein R is the measured fluorescence ratio (I_{λ1}/I_{λ2}), where hi is the fluorescence intensity from the first fluorophore at wavelength λ1 and I_{λ2} is the fluorescence intensity from the second fluorophore at wavelength λ2, both λ1 and λ2 have background subtracted, and Ro is the ratio in the absence of bilirubin. BT is the total bilirubin concentration and PT is the concentration of the sensor, r is the I_{λ2}/I_{λ1} ratio of the bilirubin sensitive probe's fluorescence in the absence of the second fluorophore, Kd is the equilibrium dissociation constant of the sensor and Rm is the ratio R extrapolated to infinite BT. Equation (1) is used in some embodiments rather than equation (5) of U.S. Pat. No. 9,529,003 when Rm is >0.

Embodiments provided herein are directed to methods of measuring the concentration of free bilirubin [Bf] by a combination of the following steps which include optionally measuring the fluorescence of the sample, mixing the sensor with the sample, and measuring the fluorescence, optionally, subtracting the sample's fluorescence intensities (background or blank) in the absence of the sensor from sample fluorescence intensities with the sensor present, calculating R from the background subtracted sensor intensities and determining the concentration of [Bf] from equation (2).

In some embodiments, equation 2 is used to calibrate the sensor and/or measure Bf wherein R is the measured fluorescence ratio ((I_{λ1}/I_{λ2}), I_{λ1} is the fluorescence intensity from the first fluorophore at wavelength λ1 and I_{λ2} is the fluorescence intensity from the second fluorophore at wavelength λ2, both intensities with sample blank subtracted, Ro is the ratio in the absence of bilirubin, r is the I_{λ2}/I_{λ1} ratio of the sensor in the absence of the second fluorophore, Kd is the dissociation constant, Rm is the minimum R value at ∞ Bf and Rm is the R at bilirubin saturation of the probe.

In some embodiments, the sensor is composed of two fluorophores by a combination of a bilirubin responsive iLBP with one fluorophore and a second fluorophore is attached to a polymer free in solution or protein free in solution, which does not bind bilirubin. In some embodiments, a sensor is composed of a protein with one fluorophore which binds or responds to bilirubin and is attached to a solid substrate and a second fluorophore is attached to another protein that does not bind or respond to bilirubin that is also attached to the solid substrate but is separated from the protein with the first fluorophore.

In some embodiments, the sample comprises a carrier macromolecule for the bilirubin such as albumin, lipid binding proteins, lipid vesicles or cyclodextrin.

In some embodiments, the bilirubin responsive and non-responder probes are attached to the channel of a disposable microfluidics channel allowing measurements of Bf in undiluted blood samples.

In some embodiments, the sample is from a human, an animal or a plant. In some embodiments, the sample is from whole blood, blood plasma, blood serum, urine, CSF, saliva, gastric juices, interstitial fluid or lymph. In some embodiments, the sample is from patients receiving intravenous infusion of oil emulsions. In some embodiments, the sample is from patients that may be producing, from disease or stress, molecules that displace bilirubin from albumin. In some embodiments, the sample is from patients that are being treated with drugs than may displace bilirubin from albumin. In some embodiments, the sample is from patients that are undergoing phototherapy, transfusion or other therapies that reduce bilirubin levels.

Embodiments provided herein are directed to a kit, which may include one or more collection devices for collecting a sample from a patient, one or more sensors as described above or a composition containing one or more sensors in a suitable carrier, and optionally, reference standards comprising a known concentration of unbound bilirubin.

Embodiments provided herein are directed to probes as defined in any of Tables 1 or 2.

For purposes of the present disclosure bilirubin is the Z,Z isomer of unconjugated bilirubin IXα [McDonagh A F et al. Photoisomers: obfuscating factors in clinical peroxidase measurements of unbound bilirubin? Pediatrics 123: 67-76, 2009]. Unbound bilirubin is the aqueous monomer cfunconjugated Z,Z IXα bilirubin as distinct from bilirubin that in blood plasma is generally found bound to albumin.

For the purposes of the present disclosure, the term “lipid” is taken to have its usual and customary meaning and defines a chemical compound which is most soluble in an organic solvent but has some level of solubility in the aqueous phase (the fraction that is unbound). Accordingly, a “lipid-binding protein” includes any protein capable of binding a lipid as lipid is defined herein.

Levels of unbound molecules, such as for example bilirubin, lipids, including fatty acids, hormones, and metabolic products, can provide information diagnostic of health and disease when measured in appropriate human or animal fluids. It is increasingly apparent that determination of the unbound (also referred to herein as ‘aqueous phase’ or ‘free’) concentration of such molecules provides critical information about physiologic homeostasis. Many metabolites are hydrophobic molecules with low aqueous solubility and unbound concentrations that are much lower than their “total” concentration, where the bulk of the “total” may be bound to proteins or cells. In biological fluids the concentration of the unbound molecules is often regulated to maintain a relatively constant unbound concentration under normal physiologic conditions. This regulation occurs through the interaction of the molecules with a carrier protein such as for example, albumin. Thus, most of the molecules are generally bound to albumin, or other carriers. However, a small fraction of the molecules may dissociate (and rebind) from the albumin into the aqueous phase and these are the unbound molecules.

For the purposes of the present disclosure, in some embodiments “bilirubin sensors” are two iLBPs that are labeled with two different fluorophores at cysteine residues and where the first iLBP undergoes a change in a fluorescence index upon binding bilirubin and the second iLBP does not significantly change its fluorescence in the presence of bilirubin (a non-responder probe). In some embodiments, a bilirubin sensor may also comprise an iLBP fluorescently labeled at a cysteine residue with additional fluorescence provided by a second fluorophore that may be free in solution, attached to a different molecule or polymer and the second fluorophore does not change fluorescence in the presence of bilirubin. In this case, if the fluorescence of only one of the fluorophores changes upon binding bilirubin the ratio of fluorescence indices at 2 wavelengths will be different. Such probes may be used to determine specifically the aqueous concentration of unbound bilirubin, which is otherwise difficult because of its poor solubility properties in aqueous solutions and because of the presence of other metabolites especially free fatty acids. A change in the ratio of the fluorescence response is especially important for the accurate determination of the intracellular concentrations of unbound bilirubin and is important for improving the accuracy and precision of the determination of the extracellular concentrations of unbound bilirubin.

U.S. Pat. Nos. 7,601,510, 9,134,317 and 9,529,003, [Huber A H et al. Fatty acid-specific fluorescent probes and their use in resolving mixtures of different unbound free fatty acids in equilibrium with albumin. Biochemistry 45: 14263-14274, 2006] and [Huber A H and Kleinfeld A M. Unbound free fatty acid profiles in human plasma and the unexpected absence of unbound palmitoleate. J. Lipid Res. 58: 578-585, 2017] describe methods for the high throughput generation of highly specific probes that allow for the determination of unbound analytes. U.S. Pat. Nos. 7,601,510, 9,134,317 and 9,529,003 describe the development of bilirubin specific probes. Embodiments provided herein relate to improvements in the bilirubin technology that was described in U.S. Pat. Nos. 7,601,510, 9,134,317 and 9,529,003. Embodiments of the present disclosure improves the accuracy and precision for the determination of unbound bilirubin levels and allows the technology to be used in different instrumentation formats. The bilirubin probes described in U.S. Pat. Nos. 7,601,510, 9,134,317 and 9,529,003 and [Huber et al. Fluorescence Sensor for the Quantification of Unbound Bilirubin Concentrations. Clin Chem 58: 869-876, 2012] were primarily methods that used the probes in aqueous suspension (cuvette based fluorometry) and primarily used diluted plasma or serum samples. Embodiments disclosed herein relate to the methods of generating Bf sensors by using two probes, iLBP muteins that are labeled with fluorophores that fluoresce in the near infrared. The first iLBP is labeled with LICOR 700DX-maleimide, which fluoresces at 700 nm and its fluorescence is quenched upon binding Z,Z bilirubin. The second iLBP is labeled with LICOR 800CW-maleimide, which fluoresces at 819 nm and does not respond to bilirubin. Both the LICOR 700DX-maleimide and the LICOR 800CW-maleimide can be excited at 660 nm. Some embodiments provided herein relate to quenching of long wavelength probes by bilirubin. In addition, some embodiments relate to methods of attaching bilirubin probes to solid surfaces and using such compositions for measuring unbound bilirubin in microfluidics devices and disposable sample cartridges.

Bilirubin sensors are used to determine unbound bilirubin levels in blood samples and fatty acids are the most abundant metabolite in blood that have properties similar to bilirubin. Fatty acids compete with bilirubin for binding to albumin and have unbound concentrations that are similar to unbound bilirubin. Bilirubin sensitive probes are developed starting with iLBP mutants that generally have high affinity for fatty acids. Thus, the first step in discovering bilirubin probes from iLBP mutein probes is the screening of more than 300,000 such probes with up to 11 of the most abundant fatty acids to identify probes that do not significantly respond to fatty acids. Systematic high throughput screening of mutant proteins is not possible because in general mutant proteins without a fluorescence label do not elicit a measurable signal upon analyte binding. More than 10,000 such fatty acid non-responders (“non-responder library”) were identified by the finding that ΔR/ΔR_{ADIFAB}<0.1. This quantitative benchmark indicates that the affinity of these probes for fatty acids are in general at least 10-fold smaller than for ADIFAB2 reference probe. Screening these non-responder probes with bilirubin identifies potential bilirubin probes and/or templates that are used for the generation of new mutein probe libraries by further mutagenesis of the newly identified template proteins. The generated library is screened for response to fatty acids and bilirubin and the probes identified as most responsive to bilirubin and least responsive to fatty acids are either identified as bilirubin probes or may be used as templates for further rounds of mutagenesis and screening. This has been done for all the bilirubin and non-responder muteins described herein (for example, in Tables 1 and 2) and the resulting bilirubin sensors have no significant response to fatty acids.

Bilirubin probes identified by these methods to have useful properties, including a significant response to bilirubin and zero to low response to fatty acids are further characterized. A probe that does not respond significantly to FFA means that binding to FFA is more than 10 times less than binding to bilirubin. In some embodiments, binding to FFA is 100 times less than binding to bilirubin. This includes calibration to determine probe bilirubin binding affinity and fluorescence characteristics, as well as monitoring unbound bilirubin levels in aqueous solutions containing bilirubin and human serum albumin to identify potential competition with fatty acids. Non-responder probes may be generated in which fatty acids bind to the probe but do not generate a change in fluorescence. In this case fatty acids in blood samples might compete with bilirubin for binding to the probe and thereby result in an inaccurate determination of the unbound bilirubin levels. Competition with fatty acids is evaluated by determining whether the fluorescence response of a bilirubin probe plus bilirubin is altered by addition of fatty acids.

Bilirubin probes found by the methods described herein, which yield accurate bilirubin concentrations in solutions with bilirubin and albumin and, which do not exhibit detectable fatty acid competition are selected for further testing in human blood samples. Blood plasma samples from individual neonates and adult donors, as well as, pooled samples from commercial sources are used to determine whether the bilirubin probes provide accurate serum or plasma unbound bilirubin concentrations in samples that have essentially unknown levels of non-bilirubin analytes commonly present in human blood samples. Healthy adults have low bilirubin levels and bilirubin:HSA mole ratios are less than 0.1, and therefore their Bf concentration approaches zero (<1 nM). Blood samples are spiked with bilirubin and albumin concentrations are measured to obtain well-defined bilirubin:albumin ratios that yield Bf levels above the limit of detection (LOD) of assay which is less than 1 nM. The concentration of unbound bilirubin in the various samples is then measured with the Bf sensor and the results are compared with unbound bilirubin concentrations determined with the peroxidase assay [Jacobsen J and Wennberg R P. Determination of unbound bilirubin in the serum of newborns. Clin Chem 20: 783, 1974] as implemented using the only FDA cleared test for Bf, the Arrows UB-2 analyzer [Nakamura H and Lee Y. Micro determination of unbound bilirubin in icteric newborn sera: an enzymatic method employing peroxidase and glucose oxidase. Clinica Chimica Acta, 79 (1977) 411-417]. Equivalence of the Bf sensor determined plasma unbound bilirubin concentrations with those determined with the peroxidase assay confirms that blood components other than unbound bilirubin have no detectable effect on the probe performance.

U.S. Pat. Nos. 7,601,510 9,134,317 and 9,529,003 require the previously necessary and time-consuming step of characterization of bilirubin binding to the protein to be omitted; only the probe itself is characterized. This was required not only for the avoidance of the protein characterization but also because the properties of the probe are often not predictable from the ligand-protein binding characteristics. For example, different proteins can have very similar binding affinities, and yet the fluorescence response of their derivative probes can vary.

Most bilirubin probes described previously were labeled only with acrylodan, primarily at lysine 27 of SEQ ID NO: 1 (including in U.S. Pat. Nos. 7,601,510, 9,134,317 and 9,529,003 and [Huber et al. Fluorescence Sensor for the Quantification of Unbound Bilirubin Concentrations. Clin Chem 58: 869-876, 2012]). Additional bilirubin probes were labeled with two different fluorophores, acrylodan at lysine 27 of SEQ ID NO: 1: and Texas red maleimide at the cysteine of an N terminal adduct in two versions, one without and the other with the KR14 (“KR14” is an abbreviation that refers to the mutation of the following 14 surface lysines to arginine in SEQ ID NO: 1, including 7R 16R 20R 29R 37R 46R 50R 88R 92R 94R 100R 125R 129R and 130R, and has a sequence as set forth in SEQ ID NO: 2) substitution that reduces multiple acrylodan labeling. These probes have good affinities and responses to bilirubin and were not significantly affected by non-bilirubin metabolites in human blood samples. However, the acrylodan-only probes may be adversely affected by bilirubin-mediated excitation inner filter effect in samples with high bilirubin concentrations, a condition in severe neonatal hyperbilirubinemia [Bhutani V K and Johnson L. The Jaundiced Newborn in the Emergency Department: Prevention of Kernicterus. Clin Ped Emerg Med 9:149-159, 2008], and by the presence of hemoglobin in the blood sample. Double labeled probes with acrylodan plus a longer wavelength fluorophore, such as Texas Red, have severely reduced acrylodan fluorescence intensities due to energy transfer between acrylodan and the secondary fluorophore, making such probes unable to obtain accurate values for Bf in the range of clinically important concentrations.

To overcome these deficiencies, embodiments provided herein relate to sensors and methods for determining unbound bilirubin levels. Mutein libraries were identified in which the fluorophore quenched by bilirubin labels a single cysteine side chain and the position of this side chain is found to be important for optimizing the fluorescence change upon bilirubin binding. Also described are bilirubin probes in which the fluorophore quenched by bilirubin labels different cysteine side chains and where the position of the side chains is found to be important for optimizing the fluorescence change upon bilirubin binding. Also described are bilirubin quenchable fluorophores that absorb and emit at long wavelengths where bilirubin quenching by Förster type energy transfer should not occur. The disclosure also relates to fluorescence quenching by bilirubin of very long wavelength fluorophores including those extending into the infrared. Because of their long wavelength absorbance and fluorescence, such fluorophores are unaffected by bilirubin or hemoglobin absorbance or that of virtually any other chromophore potentially present in blood samples.

Additional embodiments described herein relate to methods to generate bilirubin ratio sensors that are generated with a single fluorophore on a bilirubin binding protein and whose fluorescence is reduced upon binding bilirubin and with a second different fluorophore attached to a protein that is not responsive to and/or does not bind to bilirubin (a non-responder probe). Such a sensor responds to bilirubin binding to the protein portion of the probe with a change in the ratio of a fluorescence index measured at two different wavelengths. This type of ratio sensor, one that uses an independent second fluorophore, eliminates the problem of energy transfer quenching between fluorophores that is typically observed when both fluorophores are located on the same macromolecule such as a protein. Such quenching greatly reduces the signal intensity and thereby diminishes the accuracy and precision of the measurement of the unbound bilirubin concentration. This avoidance of energy transfer is accomplished by not attaching both fluorophores to the same probe molecule.

Also described are bilirubin probes that can attach to solid substrates such as polystyrene or latex beads and, which beads can be immobilized on a surface for use in disposable microfluidics devices. The first and second fluorophores can be attached respectively to a bilirubin responsive iLBP and a bilirubin non-responder iLBP. In some embodiments, both probes can be attached to the same or different solid bead substrates so that the two fluorophores remain sufficiently separated so that energy transfer is not significant, and these beads can be immobilized on disposable plastic microfluidic devices. A mixture of the sensor-bead complexes in an aqueous buffer (slurry) is dispensed into the channel of the microfluidic devices at volumes less than 2 μl and allowed to dry so that a circular spot of diameter of approximately 2 mm is formed and adheres to the bottom of the channel. An undiluted blood samples is applied to the entrance of the channel and rapidly flows across and reconstitutes the dried bilirubin sensor spot. The blood sample containing device is then placed in a fluorescence reader which measures the ratio of fluorescence from the bilirubin responsive and non-responder probes (sensor), from which the Bf concentration is calculated.

Also described are methods for calibrating and using a bilirubin sensor described herein so that Bf concentrations can be determined in approximately 5 μl of undiluted blood samples in a single step. These small volume blood samples are applied to the disposable plastic microfluidic cartridges containing the dried bilirubin ratio sensor. The bilirubin sensor containing cartridges are preferably used for a single measurement and the calibration parameters (Kd, Rm, Ro) must be determined within a “lot” of identically manufactured cartridges. The calibration is performed by titrating a sufficient number of cartridges with aqueous samples of increasing well-defined Bf levels. Because plastics bind unbound bilirubin and because surface to volume ratio in the microfluidics channel is large, the calibration Bf samples must be highly buffered by complexes with albumin. The Bf concentrations of each complex are determined by measuring each by cuvette fluorometry using a calibrated sensor free in aqueous solutions. The free sensor is calibrated by titration with aqueous solutions of bilirubin where the concentrations of these unbound bilirubin solutions are determined by absorbance. The free probe's response (R values versus Bf concentrations) are used to determine the free sensor's calibration (Kd, Rm, Ro) (equation 1). The calibrated free sensor mixed with samples of each of the BR:HSA complexes is used to determine the Bf concentrations of each complexes by cuvette fluorometry using equation 2. These calibrated complexes are used to determine the binding parameters of the disposable cartridges including the equilibrium dissociation constant (Kd), the minimum fluorescence ratio (Rm) and the initial ratio (Ro) under defined conditions, of temperature, pH, and solution composition, suitable for blood samples. Binding isotherms are performed in aqueous buffer by measuring the change in fluorescence of the bilirubin sensor in response to increasing bilirubin concentrations (“titration data”). The set of fluorescence responses at each bilirubin concentration are fitted with an appropriate equation (“calibration equation (3)”) that correctly describes the fluorescence response as a function of the Bf concentration, the, specific spectroscopic characteristics, and the Kd.

$$R = \frac{{K}_{d} ( {rR}_{m} - 1 ) {R}_{o} + {B}_{f} ( {rR}_{o} - 1 ) {R}_{m}}{{K}_{d} ( {rR}_{m} - 1 ) + {B}_{f} ( {rR}_{o} - 1 )} \qquad ( 3 )$$

Free bilirubin concentrations ([Bf]) are determined in samples in which [Bf] is buffered by the albumin binding equilibrium and is therefore not significantly perturbed by the presence of the bilirubin probe. Equations (2) is used to determine [Bf] for ratio sensors in which Rm is >0.

Some embodiments provided herein relate to the development of fluorescent protein molecules that can be used to determine the concentration of unbound analytes. More particularly, some embodiments relate to 1) identification of bilirubin probes generated by the methods of U.S. Pat. Nos. 7,601,510, 9,134,317 and 9,529,003, which are expressly incorporated herein by reference and modifications of these methods are also described, 2) use of such probes for clinical medicine and basic science, or 3) examples of these probes for the determination of the unbound bilirubin concentration in different fluids.

Bilirubin probes are iLBP proteins that have been ‘labeled’ through the covalent addition of one or more fluorescent molecule(s) (fluorophore(s)) that exhibit a change in a fluorescent index upon binding bilirubin. In some embodiments, the probe contains a single cysteine to which a fluorophore is covalently attached.

In some embodiments, two different fluorophores are used, where one of the two fluorophores labels an iLBP that is responsive to bilirubin binding and demonstrates a change in a fluorescence index upon binding of bilirubin to the probe. The second fluorophore labels an iLBP that is not responsive to or does not bind bilirubin. The second probe provides a reference point so that a difference in ratio of fluorescence at two different wavelengths is observed upon bilirubin binding. The second probe may not react to the bilirubin binding or may react in a different manner from the first fluorophore. In some embodiments, the second fluorophore has an emission point at a different wavelength relative to the first fluorophore. Examples of chemical dyes, which may be used as a second fluorophore include, but are not limited to, LI-COR 800CW maleimide, Cy7 maleimide, Cy7.5 maleimide, VivoTag-S 750-M, and Alexa Fluor 750. In some embodiments, the second fluorophore is LI-COR 800CW maleimide.

In some embodiments, two different fluorophores are used in which one fluorophore is attached to a cysteine and this fluorophore is responsive to bilirubin binding, that is, demonstrating a change in a fluorescence index upon binding of bilirubin to the fluorescently labeled iLBP mutein, The second fluorophore is not chemically linked to the bilirubin binding iLBP mutein and is not sensitive to bilirubin binding to the iLBP mutein. The second fluorophore provides a reference point so that a difference in ratio of fluorescence at two different wavelengths is observed upon bilirubin binding. In one embodiment the second fluorophore has an emission point at a longer-wavelengths relative to the first fluorophore. Examples of chemical dyes which may be used as the first fluorophore include but are not limited to LI-COR 700DX maleimide, Biotum CF680-M, CF680R-M, Lumiprobe Cy5, Cy7, Perkin Elmer VivoTag 645-M, VivoTag 680XL-M, Atto Tek Atto 680, Atto 700, Dyomics DY677, or DY 689. Examples of chemical dyes which may be used as a second fluorophore include, but are not limited to, LI-COR 800CW maleimide. In some embodiments, the first fluorophore is LI-COR 700DX maleimide and the second fluorophore is LI-COR 800CW maleimide.

The iLBP muteins may be “tagged” so that they bind to a solid support with high affinity. This includes but is not limited to tagging with biotin, Flag-epitope or c-myc epitope or HA-tag, glutathione-S-transferase (GST), maltose binding protein (MBP), a chitin binding domain (CBD), Thioredoxin, β-Galactosidase, VSV-Glycoprotein, calmodulin binding protein, a polystyrene (PS) hydrophobic tag or a metal affinity tag such as a 6× His tag. The specific association of the affinity tag with the solid support material facilitates unbound bilirubin measurements in flat surface configurations including but not limited to multi-well plates and microfluidics devices. By virtue of their attachment to solid supports the probes can be concentrated in a restricted, effectively two-dimensional region. This enables measurements of unbound bilirubin within thin layers of sample solutions allowed to flow across the probes, which are confined to the effectively two-dimension region. This effectively allows for front surface fluorescence measurements which reduces absorbance due to bilirubin and hemoglobin and facilitates measurements in whole blood. The affinity tag(s) may be fused at either the NH2- or COOH- termini or at both termini simultaneously, as shown for example in Tables 1 and 2. In some embodiments, a 6× Histidine tag was fused to either the NH2- or COOH— termini of the iLBP mutein or at both termini simultaneously without significantly changing the protein's bilirubin binding properties. In some embodiments, the fusion peptide is composed of two separated Histidine regions at the COOH terminus of the probe. In some embodiments, the probes are immobilized on a solid support including but not limited to Ni-polystyrene beads.

In some embodiments the bilirubin sensor immobilized on a solid support is a combination of two probes labeled with different fluorophores that excite at the same wavelength but emit at two different wavelengths. One of the two probes is responsive to bilirubin binding that is, it demonstrates a change in a fluorescence index upon binding of bilirubin to the protein. The second probe labeled with a different fluorophore is not responsive to binding of bilirubin or changes differently than the first probe in response to bilirubin. The second probe's fluorophore provides a reference point so that a difference in ratio of fluorescence at two different wavelengths is observed upon bilirubin binding. In some embodiments, the first, bilirubin sensitive, protein is labeled with LI-COR 700DX maleimide and the second bilirubin insensitive protein is labeled with LI-COR 800CW maleimide.

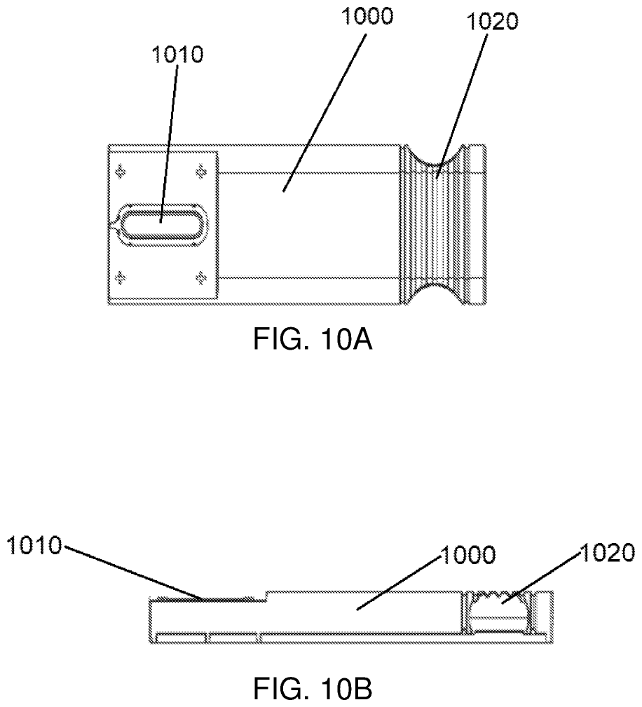

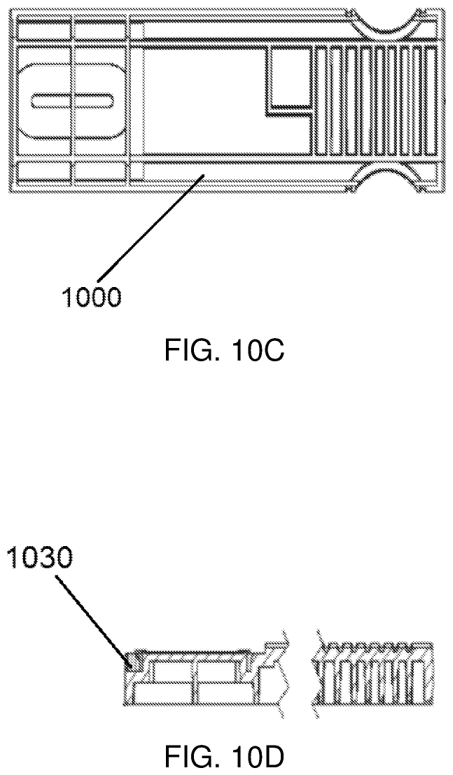

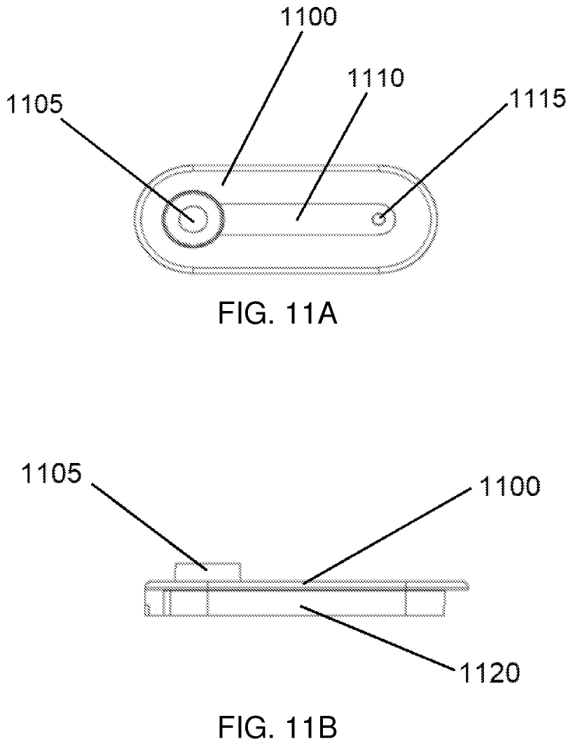

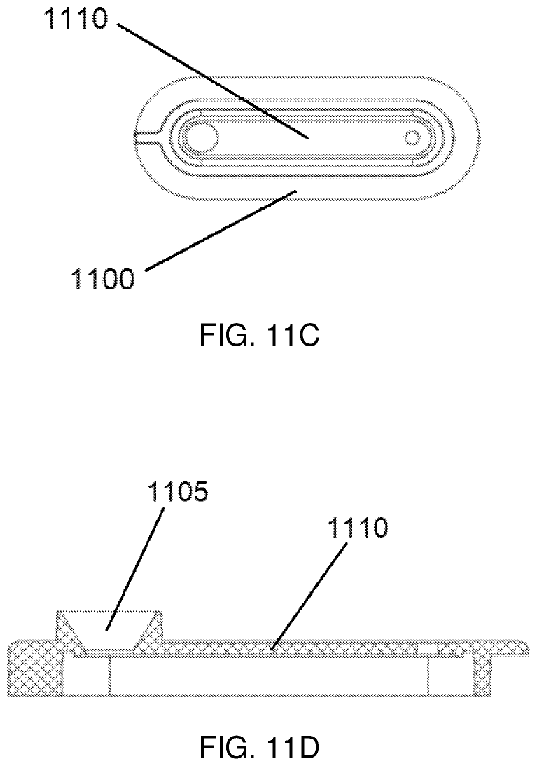

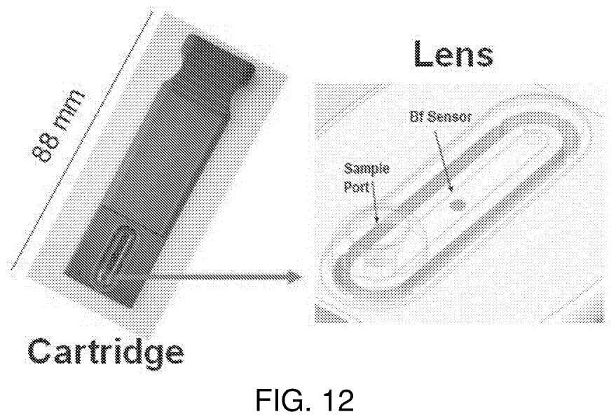

Some embodiments provided herein relate to disposable sample cartridges containing the Bf sensor, as depicted in FIGS. 10A-10D, 11A-11D, and 12. After the sample is added, the cartridge is placed into a fluorescence reader to determine the Bf concentration of the sample. In some embodiments, the cartridge is composed of a polystyrene base and an acrylic lens as in FIGS. 10A-10D and 11A-11D. In some embodiments, the sensor is composed of a bilirubin sensitive iLBP from Table 1, LICOR 700DX-maleimide and a non-responder iLBP from Table 2, LICOR 800CW-maleimide, in which the two probes are bound to Ni-NTA polystyrene beads (e.g. dynal 1 μm NTA beads) either on separate beads or on the same beads. Some embodiments add a peptide from Table 4 to the Ni-NTA polystyrene beads with bound probes to help reduce interference from hemoglobin and/or hemolysate. The probes and peptide labeled beads are suspended in aqueous buffer to form a slurry, which is dispensed as a drop of 250 to 2000 nL volume on to the polystyrene base. This spot of Bf sensor beads is allowed to dry and is then enclosed by the acrylic lens that forms a channel containing the dried sensor spot. In some embodiments, the polystyrene base is treated with UV irradiation prior to applying the sensor slurry. In some embodiments, the UV irradiation at about 185 nm wavelength is superior to 254 nm irradiation for anchoring the polystyrene bead-sensor and forming well defined circular sensor spots on the polystyrene base. In some embodiments, after the sensor spot is dried, the plasma treated acrylic lens is snapped on to the polystyrene base forming a sealed channel of 2.7 mm in width, 0.1 mm in height and 13.7 mm in length (total volume=3.7 μl). The sample is then applied to the sample port of the lens which rapidly fills the channel and reconstitutes the dried sensor.

In some embodiments, the cartridge is a microfluidic device, and includes a polystyrene base; an acrylic lens; and a substrate having a bilirubin responsive probe, a non-responder probe, and anti-hemoglobin peptides immobilized thereto. In some embodiments, the polystyrene base includes a material having a dark color configured to reduce reflection intensity of exciting light at 660 nm, such as a dark gray base. In some embodiments, the substrate are beads, to which the probes and/or anti-hemoglobin peptide is attached. In some embodiments, the beads are applied as a slurry in a less than 2 μL volume to a defined region on the base, and then allowed to cure, such as by phototreatment. In some embodiments, the base is treated with UV light at a wavelength ranging from about 145 nm to about 225 nm, thereby phototreating polystyrene polymer chains with polymer chains of the substrate. In some embodiments, following curing (phototreatment), the dried sensor forms a spot on the cartridge, such as a spot having a diameter of about 2 mm. In some embodiments, the lens is placed in position on the base, and forms a channel from the sample port to a few mm beyond the sensor (FIGS. 10A-10D, 11A-11D, and 12). In some embodiments, the microfluidic device (cartridge) is placed into a Steriflex W1F pouch with desiccant.

FIGS. 10A-10D depict multiple views of an embodiment of a base of a cartridge. FIG. 10A depicts a top view of the cartridge base 1000. The cartridge base 1000 includes an open region 1010 that includes a substrate. The cartridge base 1000 may further include housing, which may include ribs 1020 for handling, including for grasping and/or for insertion or removal of the cartridge into a reader device. The cartridge base 1000 may be prepared from any suitable material, including, for example polystyrene. In some embodiments, the base 1000 includes a material having a dark color configured to reduce reflection intensity of exciting light at 660 nm, such as a dark gray base. In some embodiments, open region 1010 of the base is treating with UV light at a wavelength ranging from about 145 nm to about 225 nm. In some embodiments, the phototreatment makes polystyrene polymer chains photo labile for reaction with a substrate. In some embodiments, following phototreatment, a substrate is contacted with the phototreated region of the base, which binds the substrate to the base at a specified region within the open region 1010. The substrate may include any substrate disclosed herein.

FIG. 10B depicts a side view of the cartridge base 1000, having an open region 1010 upon which the substrate may be placed, and including ribs 1020. FIG. 10C depicts a bottom view of the cartridge base 1000. FIG. 10D depicts a magnified cross-section view of the open region 1010, depicting ribbing 1030 that becomes crushed upon coupling of the base cartridge 1000 to a lens.

FIGS. 11A-11D depict multiple view of an embodiment of a lens 1100 configured to couple to the cartridge base 1000. FIG. 11A depicts a top view of the lens 1100, which includes a sample port 1105, a fluid flow path 1110, and a bilirubin sensor 1115. FIG. 11B depicts a side view of the lens 1100. As shown in FIG. 11B, the lens 1100 includes a sample port 1105 and insertion portions 1120 that are configured to couple to the cartridge base 1000, form a fluid flow path 1110, and seal the cartridge to prevent leakage of fluids. FIG. 11C depicts a bottom view of the lens 1100 and fluid flow path 1110. FIG. 11D depicts a cross section view of the lens 1100, which shows the sample port 1105, and the fluid flow path 1110.

The lens 1100 is configured in size and shape to couple to the open region 1010 of the cartridge base 1000. Upon coupling, the lens crushes the ribbing 1030 of the cartridge base, thereby sealing the cartridge, thereby preventing leakage of fluid from the cartridge. In addition, coupling the lens 1100 to the cartridge base 1000 forms a fluid flow path 1110 through which fluid flows upon entering the sample port 1105 to the substrate.

In some embodiments, the fluid flow path is of a specified size and dimension for accurate measurement of bilirubin in the sample. In some embodiments, the surface to volume ratio of the fluid flow path is large, sufficient to measure bilirubin in an undiluted sample. In some embodiments, the sample is an undiluted sample, such as an undiluted sample of whole blood, blood plasma, blood serum, urine, CSF, saliva, gastric juices, interstitial fluid, or lymph. In some embodiments, the cartridge is configured to receive a sample in an amount ranging from less than 0.5 μL to greater than 10 μL, such as 0.1, 0.2, 0.3, 0.4, 0.5, 0.6, 0.7, 0.8, 0.9, 1, 2, 3, 4, 5, 6, 7, 8, 9, 10, 15, or 20 μL.

In some embodiments, the cartridge is configured to measure a sample at equilibrium. Prior measurement devices and/or systems, such as those commercially available, are capable of measuring bilirubin not at equilibrium. However, the present cartridge is configured to measure bilirubin in the sample at equilibrium.

In some embodiments, the cartridge is configured to improve accuracy of bilirubin measurement through traceability. Traceability may be implemented, for example, by using a standard bilirubin sample obtained from Sigma, which is used to calibrate the probes described herein. The calibrated probes are then used to calibrate sets of bilirubin-human serum albumin complexes. The calibrated complexes are then used to calibrate cartridge lots (as described in greater detail herein in the Examples).

Some embodiments provided herein relate to a custom Qiagen LR3 fluorescent reader into which the sample containing cartridge is inserted. The reader scans across the sensor with a 660 nm excitation light source and measures the 700 and 819 nm fluorescence intensities, including any fluorescence from the sample itself by also measuring fluorescence from before and after the sensor (the background intensities). The reader uses the measured fluorescence intensities to form the R value and calculates the Bf concentration using equation (2). Each cartridge may be stored in a sealed pouch containing a desiccant and a bar code containing the calibration parameters are printed on the pouch and scanned by the reader. After applying the sample to the cartridge and inserting it into the reader the Bf may be displayed in about 90 seconds.

### Using the Sensors

In some embodiments, the sample used for the determination of unbound bilirubin is a fluid sample derived from a human, an animal or a plant. In some embodiments, the fluid is whole blood, blood plasma, blood serum, urine, CSF, saliva, gastric juices, interstitial fluid or lymph. In other embodiments, determination of unbound bilirubin is performed within the cytoplasm of a cell by microinjecting or otherwise transfecting the sensor into the cell or is performed in the extracellular media of the cells or tissue slices.

A range for unbound bilirubin may be determined from a healthy population and deviations from this normal range may indicate disease.

Unbound bilirubin sensors with zero Rm may be calibrated and used for measurements of [Bf] as described in U.S. Pat. No. 9,529,003 and in [Huber et al. Fluorescence Sensor for the Quantification of Unbound Bilirubin Concentrations. Clin Chem 58: 869-876, 2012]. Methods for calibration and use of probes with Rₘ>0 are described herein for the first time in equations (1-3) (calibration) and calculating Bf, equation (2).

Unbound bilirubin sensors are used for measurements of Bf in patients at risk for bilirubin mediated toxicity, such as 80% of all newborns because they have insufficient liver function to eliminate excess bilirubin [Bhutani et al. Pre-discharge screening for severe neonatal hyperbilirubinemia identifies infants who need phototherapy. J Pediatr 2013;162:477- 82].

Unbound bilirubin sensors may be used for measurements of Bf in patients with hemolytic disease, in patients receiving intravenous infusions of oil emulsions, in patients receiving drugs that may displace bilirubin from albumin, as well as, in patients with diseases that can increase Bf by decreasing the binding affinity of bilirubin for albumin, such as sepsis, which is common in premature newborns and increases FFA levels [Nogueira et al. Changes in plasma free fatty acid levels in septic patients are associated with cardiac damage and reduction in heart rate variability. Shock 29: 342-348, 2008 and [Hegyi et al. Effects of Soybean Lipid Infusion on Unbound Free Fatty Acids and Unbound Bilirubin in Preterm Infants J Pediatr 2017;184:45-50].

Unbound bilirubin sensors may be used for measurements of Bf in patients receiving phototherapy, transfusion or other therapies designed to reduce bilirubin toxicity.

Because unbound bilirubin not total bilirubin is toxic, unbound bilirubin rather than total bilirubin may be monitored during phototherapy to ensure that unbound bilirubin decreases significantly. Although total bilirubin has been shown to decrease in response to phototherapy, in the presence of bilirubin displacing molecules such as FFA and certain drugs prescribed for newborns, total bilirubin and unbound bilirubin may be almost completely decoupled. Under these conditions virtually complete destruction of total bilirubin might be required to lower unbound bilirubin levels to those considered non-toxic. Thus, total bilirubin levels would be required that are much lower than currently achieved even for aggressive therapy [Hegyi et al. Unbound free fatty acids from preterm infants treated with intralipid decouples unbound from total bilirubin potentially making phototherapy ineffective. Neonatology 2013;104:184-187 and Hegyi et al. Effects of Soybean Lipid Infusion on Unbound Free Fatty Acids and Unbound Bilirubin in Preterm Infants J Pediatr 2017;184:45-50]. Moreover, the peroxidase assay cannot be used to monitor unbound bilirubin during phototherapy because this test does not distinguish between photoisomers of bilirubin or conjugated bilirubin and the “native” unconjugated IX-α (Z,Z) isomer. In contrast unbound bilirubin measured with the UBCheck sensors described in this application are specific for the native unconjugated IX-α (Z,Z) isomer.

The only method currently used for determining unbound bilirubin is based on horseradish peroxidase oxidation of bilirubin [Jacobsen J and Wennberg R P. Determination of unbound bilirubin in the serum of newborns. Clin Chem 20: 783, 1974]. Implementation of the peroxidase assay is available using an FDA approved assay (Arrows Ltd, Osaka, Japan). Adoption of this method for the general screening of jaundiced newborns has been limited because of issues with the Arrows method that complicate accurate unbound bilirubin determinations [Ahlfors C E. Measurement of plasma unbound unconjugated bilirubin. Anal Biochem 279: 130-135, 2000; Ahlfors et al. Effects of sample dilution, peroxidase concentration, and chloride ion on the measurement of unbound bilirubin in premature newborns. Clin Biochem 40: 261-267, 2007]. Importantly multiple, relatively large sample volumes (20 to 25 μL) of plasma or serum are required to measure Bf with the Arrows UB analyzer UA-2. Moreover, the Arrows assay which dilutes the sample by 52 fold does not determine the equilibrium unbound bilirubin concentration and corrections are needed for interferants using the Arrows method [Ahlfors et al. Effects of sample dilution, peroxidase concentration, and chloride ion on the measurement of unbound bilirubin in premature newborns. Clinical Biochemistry 40 (2007) 261-267; Ahlfors et al. Unbound (Free) Bilirubin: Improving the Paradigm for Evaluating Neonatal Jaundice. Clinical Chemistry 55:7 1288-1299 (2009)].

Embodiments provided herein relate to methods for measuring unbound bilirubin in a single step. In some embodiments, the method overcomes shortcomings of the peroxidase-Arrows method. In some embodiments, the methods use fluorescently labeled mutated fatty acid binding proteins (unbound bilirubin sensors) that allow direct monitoring of the equilibrium unbound bilirubin concentration in undiluted blood samples. The probes may be specific for the Z,Z isomer of the unconjugated bilirubin and bind the Z,Z isomer bilirubin with high affinity. Moreover, the unbound bilirubin sensors may be highly specific for unbound bilirubin and do not respond or bind significantly to free fatty acids (FFA), photo-isomers of bilirubin, conjugated bilirubin, other metabolites and most drugs present in blood. The unbound bilirubin sensors may be used to determine unbound bilirubin levels in jaundiced patients including neonates to evaluate the potential risk of bilirubin neurotoxicity and thereby accurately direct treatment to prevent the consequences of such toxicity.

In some embodiments, the methods further include administering a treatment or therapy to a subject selected or identified as one having or suffering from bilirubin neurotoxicity based on the outcome of risk of bilirubin neurotoxicity. In some embodiments, the treatment or therapy is phototherapy, exchange transfusion, intravenous immunoglobulin treatment (IVIg), or drug treatment with a bilirubin inhibitor. In some embodiments, phototherapy treatment or therapy includes exposure to a lamp that emits light in the blue-green spectrum, wherein exposure to light increases bilirubin excretion. In some embodiments, exchange transfusion includes repeatedly withdrawing blood and replacing it with unaffected blood, such as blood from a donor. In some embodiments, IVIg treatment or therapy includes intravenous transfusion of a blood protein than can reduce levels of antibodies in blood of a subject suffering from bilirubin neurotoxicity. Other treatments or therapies may include, for example, treatment or inhibition of an underlying cause of bilirubin neurotoxicity, including, for example, treatment or inhibition or amelioration of bile duct blockage, treatment or inhibition or amelioration of infectious causes or genetic disorders such as Crigler-Najjar and Gilbert syndrome.

### EXAMPLES

Embodiments are further defined in the following Examples. It should be understood that these Examples are given by way of illustration only. From the above discussion and these Examples, one skilled in the art can ascertain the characteristics of embodiments described herein, and without departing from the spirit and scope thereof, can make various changes and modifications to the embodiments to adapt it to various usages and conditions. Thus, various modifications of the embodiments, in addition to those shown and described herein, will be apparent to those skilled in the art from the foregoing description. Such modifications are also intended to fall within the scope of the appended claims. The disclosure of each reference set forth herein is incorporated herein by reference in its entirety, and for the disclosure referenced herein.

### Example 1

### Sequences of the Bilirubin Sensitive (Responder) Probes and the Non-Responder Probes Produced by Mutations of the Wild Type Intestinal Fatty Acid Binding Protein (SEQ ID NO: 1).

Wild type intestinal fatty acid binding protein from rat (WT rIFABP) has a sequence as set forth in SEQ ID NO: 1. Table 1 shows the Bf sensitive probes with LICOR 700DX-maleimide labeled at a single substituted cysteine from position 24 to 98 of SEQ ID NO:1 . In addition, each probe listed in Table 1 has an N terminal MGI. The Bf responsive probes have substitutions of 14 arginines at 14 accessible lysines, which are 7R, 16R, 20R, 29R, 37R, 46R, 50R, 88R, 92R, 94R, 100R, 125R, 129R and 130R (KR14) of SEQ ID NO:1. The sequence of KR14 is set forth in SEQ ID NO: 2. Responsive and non-responsive mutants have a C terminal double HIS tag linker, Arg Gly Ala Ala Ser His His His His His His Ser His Arg Ala Thr Pro Asn Thr Ser Pro His His His His His His His (C2XH11; SEQ ID NO: 3). Thus, each probe listed in Table 1 includes an N terminal MGI-KR14-C2XH11, in addition to the additional substitutions and additions shown in the table.

TABLE 1
Bf Sensitive Probes

| | Substitutions and |
|---|---|
| Probe | additions to SEQ ID NO: 1 |
| Mut: SEQ ID NO: 2--24C | MGI - KR14 - 14R 18L 24C 27A |
| | 38V 60R 73F 106L 115R 117D |
| | 131D - C2XH11 (SEQ ID NO: 4) |
| Mut: SEQ ID NO: 2--25C | MGI - KR14 - 14R 18L 25C 27A |
| | 38V 60R 73F 106L 115R 117D |
| | 131D - C2XH11 (SEQ ID NO: 5) |
| Mut: SEQ ID NO: 2--26C | MGI - KR14 - 14R 18L 26C 27A |
| | 38V 60R 73F 106L 115R 117D |
| | 131D - C2XH11 (SEQ ID NO: 6) |
| Mut: SEQ ID NO: 2--27C | MGI - KR14 - 14R 18L 27C |
| | 38V 60R 73F 106L 115R 117D |
| | 131D - C2XH11 (SEQ ID NO: 7) |
| Mut: SEQ ID NO: 2--29C | MGI - KR14 - 14R 18L 27A |
| | 29C 38V 60R 73F 106L 115R 117D |
| | 131D - C2XH11 (SEQ ID NO: 8) |
| Mut: SEQ ID NO: 2--30C | MGI - KR14 - 14R 18L 27A 30C |
| | 38V 60R 73F 106L 115R 117D |
| | 131D - C2XH11 (SEQ ID NO: 9) |
| Mut: SEQ ID NO: 2--33C | MGI - KR14 - 14R 18L 27A 33C |
| | 38V 60R 73F 106L 115R 117D |
| | 131D - C2XH11 (SEQ ID NO: 10) |
| Mut: SEQ ID NO: 2--54C | MGI - KR14 - 14R 18L 27A 38V |
| | 54C 60R 73F 106L 115R 117D |
| | 131D - C2XH11 (SEQ ID NO: 11) |
| Mut: SEQ ID NO: 2--74C | MGI - KR14 - 14R 18L 27A 38V |
| | 60R 73F 74C 106L 115R 117D |
| | 131D - C2XH11 (SEQ ID NO: 12) |
| Mut: SEQ ID NO: 2--76C | MGI - KR14 - 14R 18L 27A |
| | 38V 60R 73F 76C 106L 115R 117D |
| | 131D - C2XH11 (SEQ ID NO: 13) |
| Mut: SEQ ID NO: 2--97C | MGI - KR14 - 14R 18L 27A |
| | 38V 60R 73F 97C 106L 115R 117D |
| | 131D - C2XH11 (SEQ ID NO: 14) |
| Mut: SEQ ID NO: 2--98C | MGI - KR14 - 14R 18L 27A |
| | 38V 60R 73F 98C 106L 115R 117D |
| | 131D - C2XH11 (SEQ ID NO: 15) |

TABLE 2
Non responder probes are mutations of the wild type rat
FABP (SEQ ID NO: 1) labeled with LICOR 800CW-maleimide
at the cysteine positions shown in the left-hand column.

| | Substitutions and |
|---|---|
| Probe | additions to SEQ ID NO: 1 |
| Mut: SEQ ID NO: 1--27C | 27C 72A 73M 74F 126W 131D - |
| | C2XH11 (SEQ ID NO: 16) |
| Mut: SEQ ID NO: 1--31C | 31C 72A 73M 74F 126W 131D - |
| | C2XH11 (SEQ ID NO: 17) |
| Mut: SEQ ID NO: 1--33C | 33C 72A 73M 74F 126W 131D - |
| | C2XH11 (SEQ ID NO: 18) |
| Mut: SEQ ID NO: 1--54C | 54C 72A 73M 74F 126W 131D - |
| | C2XH11 (SEQ ID NO: 19) |
| Mut: SEQ ID NO: 1--74C | 72A 73M 74C 126W 131D - |
| | C2XH11 (SEQ ID NO: 20) |
| Mut: SEQ ID NO: 1--76C | 72A 73M 74F 76C 126W 131D - |
| | C2XH11 (SEQ ID NO: 21) |
| Mut: SEQ ID NO: 1--98C | 72A 73M 74F 98C 126W 131D - |
| | C2XH11 (SEQ ID NO: 22) |
| Mut: SEQ ID NO: 1--54C | 54C 72A 73V 74F 126W 131D - |
| | C2XH11 (SEQ ID NO: 23) |
| Mut: SEQ ID NO: 1--74C | 72A 73V 74C 126W 131D - |
| | C2XH11 (SEQ ID NO: 24) |
| Mut: SEQ ID NO: 1--76C | 72A 73V 74F 76C 126W 131D - |
| | C2XH11 (SEQ ID NO: 25) |
| Mut: SEQ ID NO: 1--98C | 72A 73V 74F 98C 126W 131D - |
| | C2XH11 (SEQ ID NO: 26) |
| Mut: SEQ ID NO: 1 A--73C | 72A 73C 74F 126W 131D - |
| | C2XH11 (SEQ ID NO: 27) |
| Mut: SEQ ID NO: 1 B--73C | 20R 27A 29R 72A 73C 74F |
| | 100R 126W 131D - C2XH11 |
| | (SEQ ID NO: 28) |

### Example 2

### Effect of Fluorophore Position on Bf Binding Characteristics and Effects of Mutations on Emission Spectra of Non-Responders Proteins

All responder probes labeled with LICOR 700DX-maleimide and non-responder probes labeled with LICOR 800CW-maleimide probes in Tables 1 and 2, respectively have qualitatively similar dependencies on Bf. The probes differ mostly in protein expression, binding parameters (Kd, Ro and Rm) and stability. Effects of different muteins and fluorophore labeling positions on Kd and Rm show that preferred responder probes may be those with low Kds and/or low Qs (Qs=Rm/Ro) which indicates the degree to which the probe is quenched by bilirubin binding (Table 3). Although the non-responders are not characterized by bilirubin binding parameters, the emission spectrum of the 800CW-maleimide is sensitive to specific substitutions that can alter the LICOR 700DX-maleimide intensities. For example, in the absence of 700DX-maleimide many initial non-responder mutants revealed a time dependent increase in emission at 700 nm relative to 819 nm. In some of these mutants the ratio of 700 to 819 nm increased from about 1% to 20% while in storage at 4° C. With suitable mutations this instability was eliminated. For example the Mut:SEQ ID NO:1 B-73C of Table 2 revealed the same 700/819 ratio (1%) at 1 and 6 days.

TABLE 3
Effect of fluorophore position on probe
response (700DXM = 700DX-maleimide).

| Probe | K_{d} (nM) | Qs (Rₘ/Rₒ) |
|---|---|---|
| 24C- C2XH11-700DXM | 44 | 0.14 |
| 25C-C2XH11-700DXM | 12 | 0.27 |
| 26C-C2XH11-700DXM | 21 | 0.36 |
| 27C-C2XH11-700DXM | 30 | 0.21 |
| 29C-C2XH11-700DXM | 29 | 0.52 |
| 30C-C2XH11-700DXM | 41 | 0.11 |
| 33C-C2XH11-700DXM | 25 | 0.17 |
| 54C-C2XH11-700DXM | 22 | 0.48 |
| 74C-C2XH11-700DXM | 16 | 0.15 |
| 76C-C2XH11-700DXM | 14 | 0.04 |
| 97C-C2XH11-700DXM | 25 | 0.29 |
| 98C-C2XH11-700DXM | 13 | 0.03 |

### Example 3

### The Change in the Fluorescence of the Bilirubin Responsive (700 nm) and Non-Responsive (800 nm) Probes and Their Ratio (700/800)

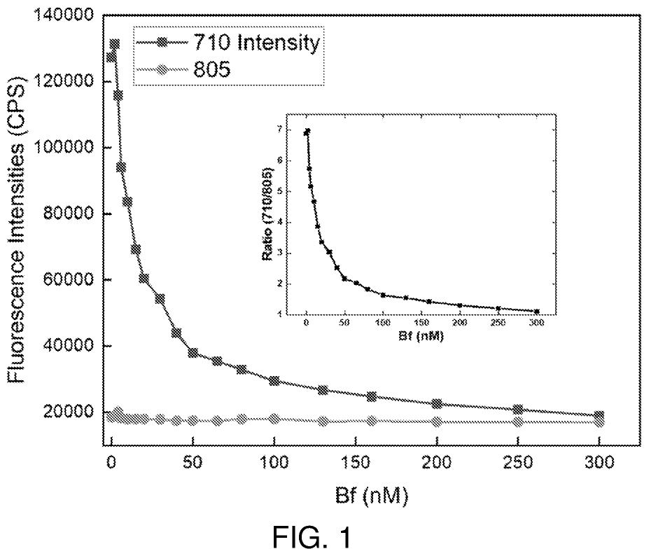

FIG. 1 depicts the intensities and ratios of intensities for a Bf sensor composed of the bilirubin responsive LICOR 700DX maleimide Mut:SEQ ID NO:2-76C probe (Table 1) and the bilirubin non-responder LICOR 800CW maleimide Mut:SEQ ID NO:1 B-73C probe (Table 2), both free in aqueous buffer, with increasing BT (total bilirubin) which at the concentrations used is primarily unbound bilirubin (Bf). Measurements of the 710 and 805 nm intensities at 675 nm excitation were performed at the probe concentrations of 1.5 nM and 10 nM, respectively in a Horiba Fluorolog 2. The results show the monotonic decrease in LICOR 700DX maleimide intensity (measured at 710 nm) attached to the bilirubin sensitive iLBP and the lack of intensity change (cv=2.5%) of the LICOR 800CW maleimide (measured at 805nm) attached to the non-responder iLBP. The insert shows the behavior of the 710/805 ratio.

### Example 4

### Calibration of the Free NIR Ratio Sensor by Cuvette Fluorometry

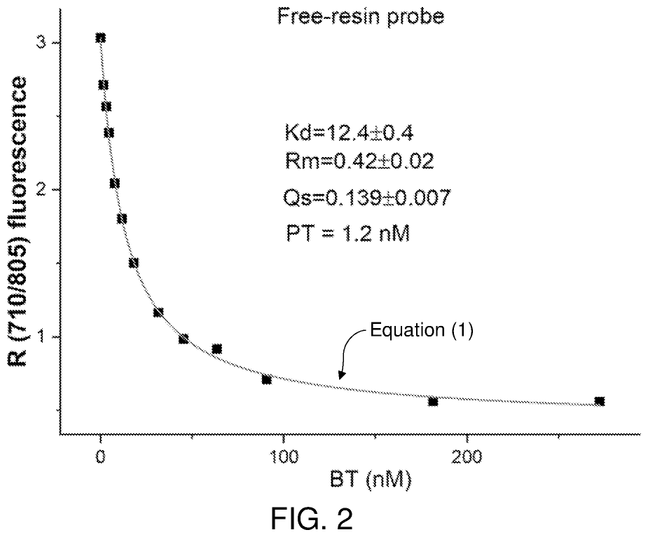

A Bf sensor composed of a LICOR 700DX maleimide labeled bilirubin sensitive iLBP (Mut:SEQ ID NO:2-76C) and a LICOR 800CW maleimide non-responder iLBP (Mut:SEQ ID NO:1 B-73C) from Tables 1 and 2, respectively, free in aqueous buffer, were titrated with increasing concentrations from a stock solution of Sigma bilirubin (Cat:B4126) solubilized in aqueous buffer at pH 12 (FIG. 2). BT concentrations were determined by absorbance at 441 nm. At each bilirubin concentration, measurements of the fluorescence excited at 660 nm and emissions at 700 nm and 805 nm were used to determine the ratio (R) of the background subtracted 700 to 805 emissions. The totalbilirubin BT in the cuvette at each step in the titration is soluble and is the total free bilirubin except for the portion that binds to the sensor. A least-squares fit to the resulting titration curve (R vs BT) (FIG. 2) is performed with equation (1), which takes account of the probe bound bilirubin. This procedure calibrates the free probe by determining its calibration parameters (Kd, Rm, and Ro). The results of the fit in this case yields a Kd of 12.4±0.4 nM, an Rm of 0.42 an Ro of 3.02 and Qs which is Rm/Ro of 0.139±0.007.

### Example 5

### Calibration of the Bilirubin-Albumin (HSA) Complexes

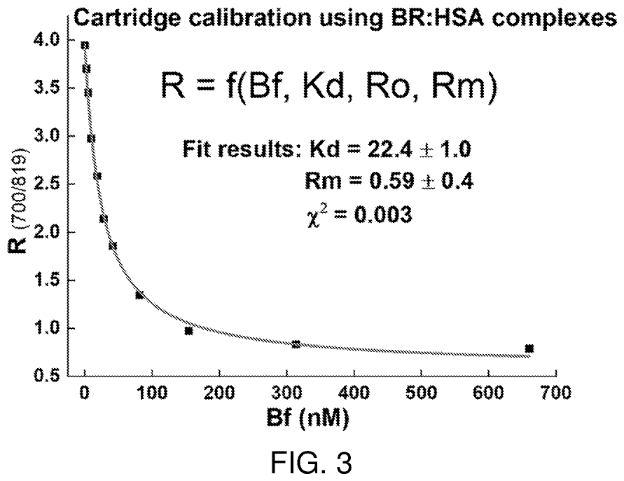

For calibrating the Bf test cartridges, a series of aqueous samples with increasing highly buffered Bf concentrations is prepared. A highly buffered Bf sample is one whose Bf concentration is not affected by bilirubin binding to the walls of the cartridge's channel nor to binding to the dried bilirubin probe in the channel. A complex of bilirubin bound to HSA (BR:HSA), with HSA at concentrations of between 400 and 1000 μM is sufficient to buffer or clamp the Bf concentrations in the test cartridge where Bf remains unchanged from the levels calibrated when bilirubin wall binding is negligible. The BR:HSA complexes are typically prepared at bilirubin to HSA at molar ratios from 0.1 to 1.0 in steps of 0.05 to 0.1. The complexes are calibrated by measuring the Bf concentration produced by each complex in cuvettes by adding to each undiluted complex a calibrated free Bf sensor as in Example 4 at approximately 1-20 nM. Typically the Bf values increase exponentially with increasing BR:HSA from 0 to 0.9 where starting from 0.1, Bf increases from 2 to 300 nM (FIG. 3).

### Example 6

### Calibration of the Test Cartridges with BR:HSA Complexes

Substantial numbers of test cartridges produced in a given period, for example 500 to 2000 or more, termed a “lot” are calibrated by selecting at random sufficient numbers of cartridges so that each bilirubin-albumin complex can be applied to replicate cartridges. A single calibration of the lot requires at least triplicate measurements of each complex, which, depending upon the BR:HSA step size, will range from 33 to 60 cartridges. Each cartridge is used only for a single measurement and disposed. The measured R values from each cartridge and each complex are fitted by least squares with equation 2 to obtain the Ro, Kd, and Rm for the cartridge lot. An example of the cartridge calibration using BR:HSA complexes reveals that the apparent the Kd and Qs can be similar to or larger than the free probe parameters (FIG. 3). The increase in Kd is may be a consequence of immobilization and interactions of probes with adjacent surfaces upon drying the probe-beads on the cartridge.

### Example 7

### One Step Disposable Cartridges

The one-step disposable cartridge is an essential component of the present invention. The cartridge includes all the components needed for a measurement of Bf upon addition of the patient's blood sample and the insertion of the cartridge into a reader. Newborn premature infants can weigh as little as 400 g and it is essential that very small blood volumes are used for the Bf determination. The minimum sample volume of the disposable cartridge is 3.8 μl. Moreover, because whole blood, as well as plasma, serum and other fluids, are to be measured, the optical path length of the excitation and emission light paths should be as small as possible. These considerations led to the development of the cartridge shown in FIGS. 10A-10D and 11A-11D. In this device, sample is added to the port and fills the channel formed by the acrylic lens that is snapped onto the cartridge base which contains the bilirubin sensor spot at the center of the channel. The resulting channel has a height of 0.1 mm, a width of 2.7 mm and a length of 14 mm which accommodates a sample volume of 3.8 μL.

High tolerances in molding are required to maintain these dimensions. An important and novel component of this device is the ability to form a sealed channel over the dried Bf bilirubin sensor-polystyrene beads by simply snapping the transparent acrylic lens on to the polystyrene base (FIGS. 10A-10D, 11A-11D, and FIG. 12). This is accomplished by the lens creating an oval grove in the base when the lens is snapped into position, forming a sealed channel by pressing against crush ribs below the surface of the base. To accomplish rapid capillary flow of the sample over the bilirubin sensor spot, the acrylic lens is treated with 02 plasma at a level that increases the hydrophilicity of the lens without compromising its optical transparency in the NIR. In addition, the polystyrene base is treated with UV radiation to increase its hydrophilicity, to enhance rapid capillary flow and to adjust the sensor spot size. The UV irradiation is also important to allow the polystyrene beads of the bilirubin sensor to tightly adhere to the polystyrene base. UV radiation at 254 nm has been shown to break the polymer bonds and thereby allow the intercalation of the polymer chains of opposing surfaces, which increases the binding affinity between the surfaces. [Maeda et al. Adhesion and friction mechanisms of polymer-on-polymer surfaces. Science (2002) 297, 379- 382]. Irradiation from 254 nm sources increases the binding affinity between polystyrene surfaces. However, the degree of adhesion is highly sensitive to the age of the 254 nm bulbs and the irradiation time can be longer than 60 minutes. An invention of this application is the discovery that typical 254 nm bulbs also produce low levels of 185 nm radiation suggesting that the 185 nm radiation is primarily responsible for the increased polymer adhesion. Switching to 185 nm sources reduces exposure times to seconds or minutes depending upon the irradiation distance. Moreover, spotting the sensor on 185 ±40 nm irradiated cartridges yields more uniform spot geometry. Accordingly, in some embodiments, the wavelength ranges from about 145 nm to about 225 nm, such as 145, 150, 155, 160, 165, 170, 175, 180, 185, 190, 195, 200, 205, 210, 215, 220, or 225 nm, or a wavelength within a ranged defined by any two of the aforementioned values.

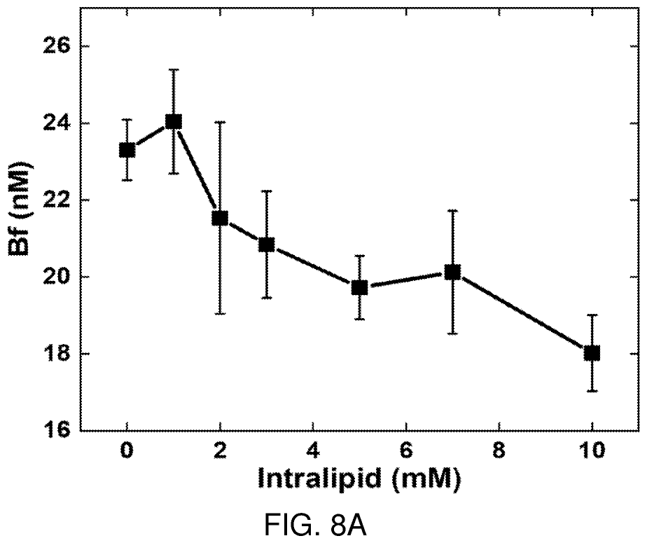

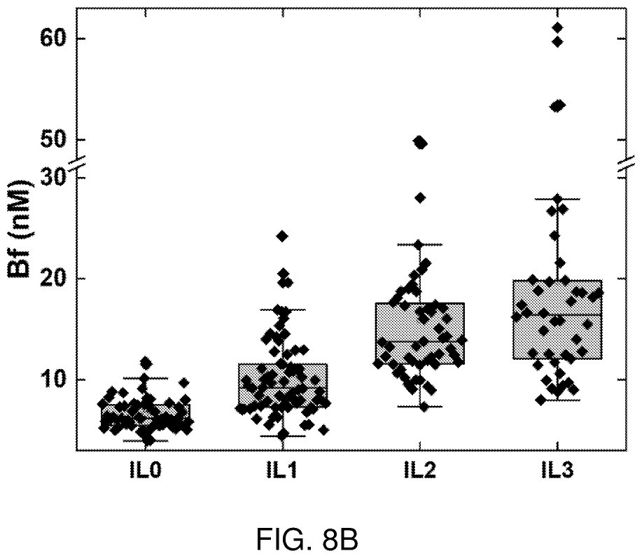

The Bf sensors attached to the cartridge surface are incompletely quenched by bilirubin, in contrast to the completely quenched sensors described in U.S. Pat. No.9,529,003. For example, U.S. Pat. No.9,529,003 describes a bilirubin probe with a HIS tag and a polystyrene tag (PS) attached to Ni-agarose beads, immobilized in the wells of “disposable cartridges”, although with no details of such cartridges. As shown in FIG. 8 of U.S. Pat. No.9,529,003 the Qs (Rm/Ro) for that probe was 0.02±0.03 (Rm=0), indicating a completely quenched Bf sensor. The finite limited quenching (Rm>0) of the present disclosure uses different analyses to determine accurate Bf levels. The analysis described herein is mathematically detailed in equations 1 and 2, which reduce to equations 2 and 3 of U.S. Pat. No.9,529,003 for Rm =0.

U.S. Pat. No.9,529,003 describes measuring Bf in whole blood in microfluidic devices in which a Bf sensor on polystyrene beads containing iron is concentrated at the bottom of multi well plates using a magnet. This configuration is not practical for a one-step assay in which the sample cartridge is sealed in a desiccant containing pouch and therefore the sensor must be tightly bound to the cartridge surface. Instead, in the current disclosure, a slurry is prepared containing the bilirubin sensitive and non-responder iLBP mutants attached to the polystyrene beads, each with different NIR fluorophores labeled at cysteine residues, all in aqueous buffer. A small drop (<2 μl) of this slurry is dispensed onto a UV treated cartridge, allowed to dry and placed in a desiccant containing pouch.

### Example 8

### Determination of the Equilibrium Bf Concentrations

Bf in blood samples is at equilibrium by virtue of the binding and dissociation of bilirubin with albumin. Typical albumin concentrations are between 400 and 600 μM and the equilibrium dissociation constant (Kd) of bilirubin for adult human albumin is about 20 nM, for the high affinity site. A bilirubin-albumin molar ratio of 0.5 at equilibrium generates a Bf of about 20 nM. The estimated amount of Bf sensor in each cartridge is 2×10⁻¹³ moles. Therefore, in a 5 μl sample the sensor concentration is 40 nM. This means, at most, the sensor would bind less than 40 nM of bilirubin or about 1.6×10⁻⁴ of the 250 μM total bilirubin bound to 500 μM albumin. Because Bf is approximately Kd*BT/albumin a small change in BT will have a negligible effect on Bf. This means that the effect of the sensor on the actual albumin buffered Bf equilibrium concentration is negligible and therefore the UBCheck approximates the equilibrium Bf concentration. It is important that the sample be undiluted for the bilirubin-albumin complexes to produce a highly buffered Bf level that remains undisturbed by the amount bound by the sensor. Other methods for measuring Bf in blood samples are not measurements at equilibrium. Most importantly the peroxidase method which has been in use for 45 years and as implemented by the FDA cleared Arrows UB Analyzer does not yield Bf at equilibrium [Jacobsen et al, Determination of unbound bilirubin in the serum of newborns. Clin Chem (1974) 20, 183]. In part this is due to the large sample dilution factor (42 to 52-fold) in the FDA cleared Arrows method and to the peroxidase method which oxidizes a substantial fraction of the Bf. A better estimate of Bf closer to equilibrium can be obtained by repeating measurements of the sample with different peroxidase concentrations and extrapolating to zero peroxidase concentration [Ahlfors et al, Unbound (Free) Bilirubin: Improving the Paradigm for Evaluating Neonatal Jaundice. Clin Chem (2009) 55:7 1288-1299]. However, this method is not FDA approved and ultimately only yields a better approximation to equilibrium. A more recent method for measuring Bf has been proposed that however also does not measure equilibrium and obtains Bf concentrations that are orders of magnitude larger than either the present invention or the results obtained with the peroxidase method. [Bell et al Paper-based potentiometric sensing of free bilirubin in blood serum. Biosensors and Bioelectronics 126 (2019) 115-121]. This method disrupts the Bf at equilibrium with albumin by using a filter and electromotive force to separate bilirubin from albumin.

### Example 9

### Effects of Dilution on Equilibrium—When Bilirubin-Albumin Displacers Increase the HSA Kd

The UBCheck most closely measures equilibrium Bf and thereby most closely approximates the steady state Bf levels in circulation. A direct illustration of the buffering power of the bilirubin:albumin complexes is the response of the sensor in the cartridge with and without albumin. Adding free bilirubin at a concentration of 100 nM to a cartridge yields ≤1 nM as detected by the Bf sensor and 50 nM Bf is detected upon adding 1000 nM free bilirubin. This loss of free bilirubin is due to its binding to the polystyrene and acrylic surfaces that define the sample channel in the cartridge, especially because of the large surface to volume ratio (20) of the sample channel.

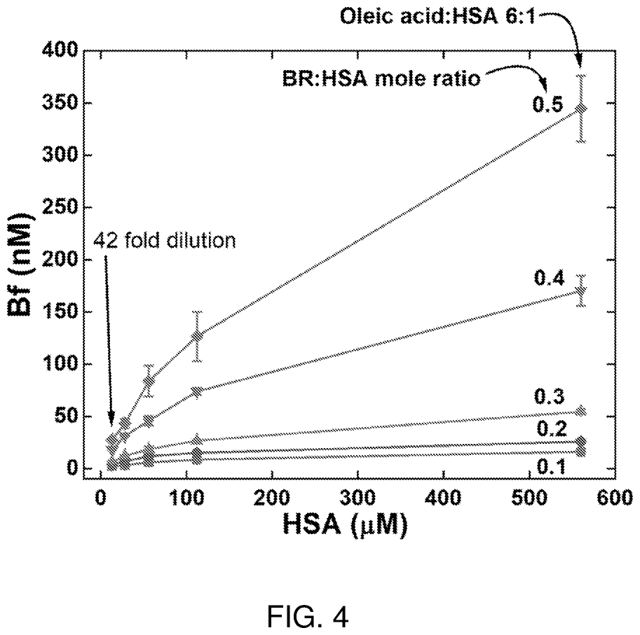

Even without binding to surfaces, equilibrium Bf concentrations depend upon sample dilution by virtue of the kinetics of the bilirubin-HSA reaction. Moreover, the effect of dilution is amplified in the presence of displacers of bilirubin from albumin which effectively reduce HSA's binding affinity for bilirubin. This is illustrated in the results of measurements of Bf as a function of bilirubin-HSA complexes at mole ratios from 0.1 to 0.5 in which oleic acid was added to HSA at a mole ratio of 6 oleic acid to 1 HSA (FIG. 4). Starting at an HSA concentration of 550 μM, the Bf concentrations decrease by more than an order of magnitude upon dilution of 42-fold (dilution used by the original Arrows UB analyzer). Oleic acid is a potent FFA displacer and is a major component of Intralipid, commonly prescribed for premature infants in the NICU.

### Example 10

### Lack of Effect of Hemoglobin/Hemolysis on UBCheck Bf Measurements

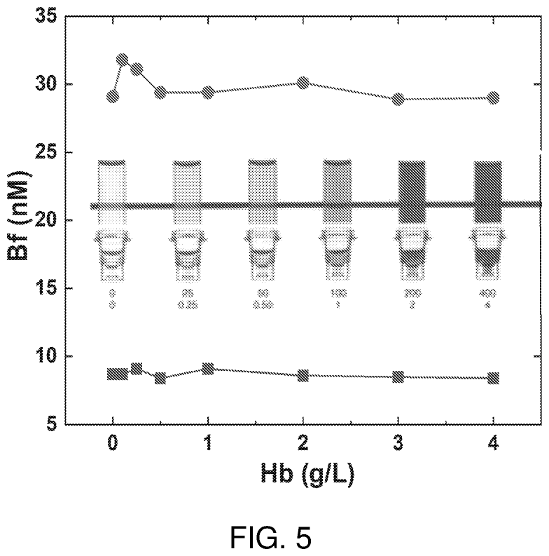

After the Bf responder and non-responder probes are applied to the Ni-polystyrene beads, the peptide of Table 4 is added to the beads and is attached through double HIS tags to the bead's Ni-NTAs. The combined probes plus peptide beads are then spotted onto cartridges. The effect of whole blood hemolysate was tested by titrating bilirubin spiked neonatal serum with increasing hemoglobin concentrations. The Bf concentrations before and after hemoglobin titration is determined and reveals that the Bf concentration relative to zero hemoglobin is unaffected by hemoglobin concentrations at least as high as 4 g/l (FIG. 5).

TABLE 4
Amino Acid Sequence of Hb blocking Peptide

(SEQ ID NO: 29)
DTETDEYAASHHHHHHSHRATTPNTSPHHHHHH

### Example 11

### Effects of Drugs prescribed to Newborn Infants

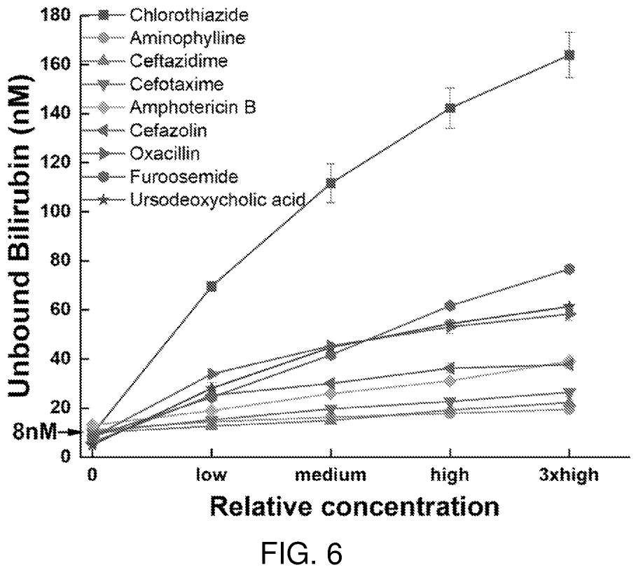

Embodiments described herein relate to measuring the concentration of Bf in newborn infants. Premature infants who are at high risk for bilirubin neurotoxicity are of particular concern because NICU infants frequently receive medications. [Hsieh et al. Medication use in the neonatal intensive care unit. Am J Perinatol. (2014) 31, 811-822]. Table 5 shows that specific drugs, among them ones that are among the most frequently prescribed in the NICU, are powerful displacers of bilirubin from albumin and interference as determined in measurements of Bf using the one-step cartridges of Example 7. The rank of the drugs indicates their frequency of use in the NICU starting with ampicillin with rank 1 being the most prescribed drug. The elements of the “displacement or interference” column is blank if the drugs had no effect. Displacers are positive and show the percent increase for the 3x drug concentration relative to no drug. The negative value for spironolactone indicates interference with the Bf sensor. As shown in Table 5 several drugs are powerful displacers and one, spironolactone is the only NICU drug found to interfere with the Bf assay. FIG. 6 shows the effect on Bf as a function of the prescribed (low, medium, high & 3×high) drug concentration of those drugs of Table 5 that are potent displacers of bilirubin from albumin.

TABLE 5
Drug Displacement of Bilirubin from Albumin

| | | Blood Conc | 3 × Blood | Displacement or |
|---|---|---|---|---|
| Medication | Rank | High (mM) | Conc (mM) | Interference |
| Ampicillin | 1 | 2.92 | 8.76 | |
| Gentamicin | 2 | 0.18 | 0.53 | |
| sulphate | | | | |
| Caffeine | 3 | 10.5 | 31.53 | |
| Vancomycin | 4 | 0.10 | 0.31 | |
| hydrochloride | | | | |
| Furosemide | 6 | 0.06 | 0.19 | 820% |
| Dopamine | 8 | 0.001 | 0.003 | |
| hydrochloride | | | | |
| Metoclopramide | 10 | 0.03 | 0.10 | |
| Ranitidine | 12 | 0.32 | 0.97 | |
| Cefotaxime | 15 | 1.12 | 3.36 | 157% |
| Acetaminophen | 16 | 1.01 | 3.04 | |
| Indomethacin | 17 | 0.01 | 0.02 | |
| Albuterol | 19 | 0.85 | 2.56 | |
| Hydrocortisone | 22 | 0.56 | 1.69 | |
| Tobramycin | 23 | 0.22 | 0.65 | |
| Erythromycin | 24 | 0.07 | 0.21 | |
| Dexamethasone | 26 | 0.01 | 0.04 | |
| Fluconazole | 27 | 0.40 | 1.20 | |
| Acyclovir | 30 | 0.91 | 2.72 | |
| Vitamin A | 31 | 0.02 | 0.06 | |
| Insulin | 32 | 0.0001 | 0.003 | |
| Ursodeoxy- | 33 | 0.26 | 0.78 | 1124% |
| cholic acid | | | | |
| Lansoprazole | 34 | 0.06 | 0.17 | |
| Spironolactone | 35 | 0.07 | 0.22 | −98% |
| Chlorothiazide | 36 | 0.69 | 2.07 | 1626% |
| Aminophylline | 37 | 0.13 | 0.40 | 76% |
| Ceftazidime | 38 | 1.07 | 3.21 | 76% |
| sodium salt | | | | |
| Epinephrine | 42 | 0.00 | 0.00 | |
| Metronidazole | 44 | 0.89 | 2.68 | |
| Oxacillin | 45 | 1.27 | 3.81 | 616% |
| Nafcillin | 46 | 1.12 | 3.37 | |
| sodium salt | | | | |
| Amphotericin B | 47 | 0.01 | 0.03 | 199% |
| Amikacin | 48 | 0.52 | 1.57 | |
| Ibuprofen | 50 | 0.49 | 1.48 | |
| Cefazolin | 51 | 1.07 | 3.21 | 524% |
| sodium salt | | | | |
| Levothyroxine | 54 | 0.0002 | 0.001 | |
| Budesonide | 56 | 0.01 | 0.02 | |
| Phenylephrine | 57 | 0.01 | 0.02 | |
| hydrochloride | | | | |
| Omeprazole | 58 | 0.03 | 0.09 | |

The increase in Bf caused by these drugs can greatly exceed the upper limit of normal for NICU infants and may have serious consequences for their health. Moreover, as in the effect of FFA (oleic acid) as described in Example 7, the effect of dilution greatly affects the degree of displacement caused by drugs. Table 6 illustrates this effect by comparing the Bf levels of adult serum spiked with bilirubin and then with cefazolin at 1.12 and 3.76 mM. Measurements of Bf were performed using the Arrows UB2 analyzer (52 fold dilution) and with the UBCheck (no dilution). As Table 6 reveals Bf in the presence of 1.12 and 3.76 mM cefazolin is about 3-fold and 5-fold larger in the UBCheck than Arrows.

TABLE 6
Effect of serum sample dilution on bilirubin
displacement by Cerfazolin.

| | UBCheck | | Arrows | |
|---|---|---|---|---|
| | UB (nM) | CV % | UB (nM) | CV % |
| Serum | 19.0 | 0.2% | 15.4 | 1.6% |
| Cefazolin 1.12 mM | 57.3 | 1.8% | 19.4 | 0.6% |
| Cefazolin 3.76 mM | 148.7 | 9.3% | 26.8 | 2.5% |

### Example 12

### Reduced Interference from Conjugated Bilirubin

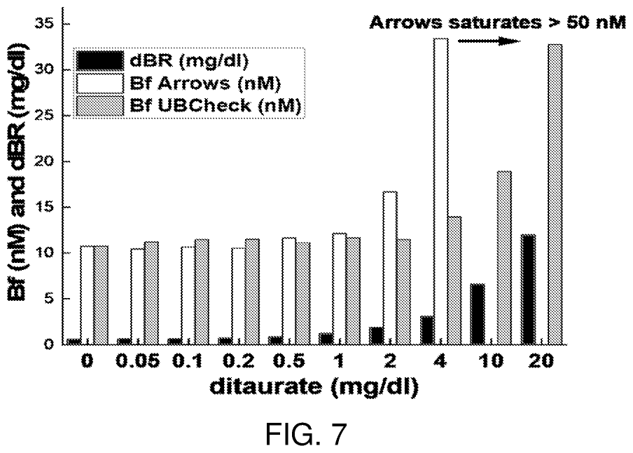

The potential interference of conjugated bilirubin with the bilirubin probes is determined by measuring the effect of ditaurobilirubin on the ability of the UBCheck to measure Bf accurately. We also compared the UBCheck results with measurements using the Arrows UB analyzer UA-2 method. The measurements are performed by spiking neonatal serum with unconjugated bilirubin to yield a Bf of approximately 10 nM and then titrating the spiked sample with ditaurobilirubin up to 20 mg/dl (FIG. 7). At each step Bf is measured by UBCheck and Arrows and the direct (conjugated) bilirubin concentration is determined using a Sigma direct bilirubin kit. At zero ditaurobilirubin both UBCheck and Arrows yield Bf of 10 nM but the direct bilirubin measurement yields a non-zero value of about 0.5 mg/dl (FIG. 7). With increasing concentrations of ditaurobilirubin UBCheck is unchanged but Arrows increases starting at about 0.5 mg/dl of ditaurobilirubin and then increases rapidly until 4 mg/dl ditaurobilirubin where the Arrow's assay saturates at Bf ≥50 nM. In contrast, the UBCheck only begins to increase at 4 mg/dl ditaurobilirubin where its Bf increase to about 12 nM, a 20% increase from the initial 10 nM level, and only at 20 mg/dl ditaurobilirubin does Bf increase to 33 nM. Thus, the UBCheck has superior specificity for Bf than Arrows even though the Arrows samples were diluted 52-fold whereas UBCheck samples are undiluted.

### Example 13

### Lack of Interference from Bilirubin Photoisomers

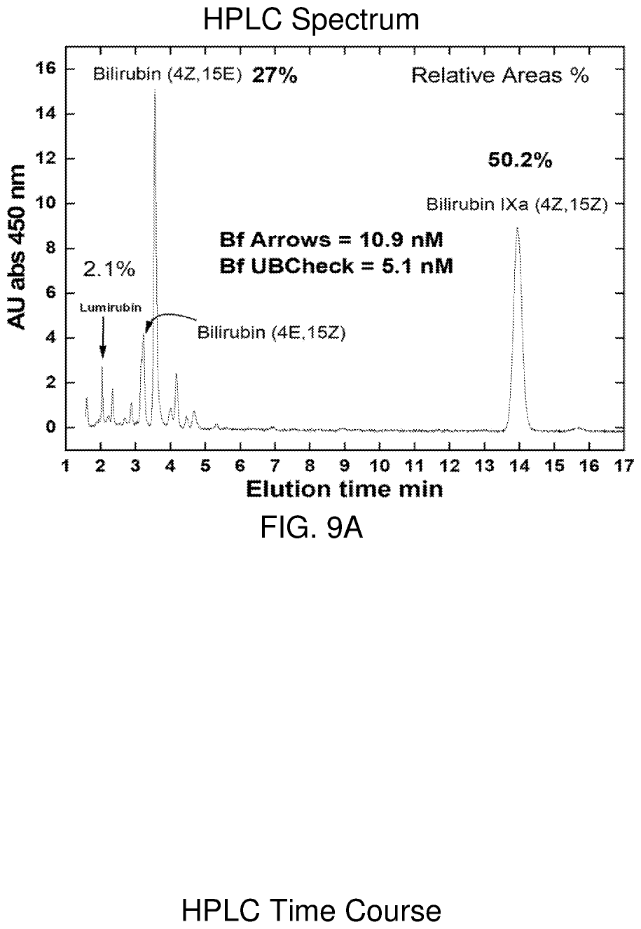

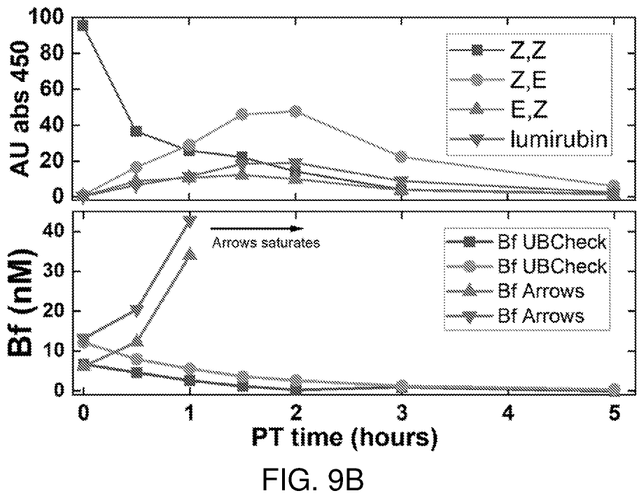

Hyperbilirubinemia in neonates is most frequently treated using blue-green light photo therapy which photo-isomerizes bilirubin bound to albumin. [Newman et al. Neonatal Hyperbilirubinemia and Long-Term Outcome: Another Look at the Collaborative Perinatal Project. Pediatrics (1993) 92, 651-657., Ennever J F. Blue light, green light, white light, more light: treatment of neonatal jaundice. Clinics in perinatology. (1990) 17, 467-481.]. The bilirubin photoisomers (4Z,15E), (4E,15Z) and lumirubin are much more soluble and therefore are readily excreted as compared to the native bilirubin IXa (4Z,15Z) molecule. Photo therapy readily reduces the Z,Z isomer and thereby efficiently treats neonatal hyperbilirubinemia. Photoisomers may also be generated by exposure to ambient light [McDonagh et al Photoisomers: Obfuscating Factors in Clinical Peroxidase Measurements of Unbound Bilirubin?. Pediatrics (2009) 123, 67-76]. As shown in FIGS. 9A and 9B, the Bf assay only detects the Z,Z isomer of bilirubin but Arrows is sensitive to Z,Z and all photoisomers. However, only the Z,Z but not the photoisomers are toxic [Jasparova et al. The Biological Effects of Bilirubin Photoisomers. (2016) PLoS ONE 11(2):e0148126.doi: 10.1371/journal.pone.0148126]. Serum samples from healthy neonates who did not receive phototherapy but for which HPLC analysis reveals substantial levels of photoisomers (presumably due to ambient light exposure of the serum) also reveal more than 2-fold larger Bf levels by Arrows compared to the UBCheck Bf assay, as shown in FIG. 9A. Adult serum samples spiked with bilirubin and then exposed to a photo therapy lamp (Natus Neoblue) for 5 hours, as shown in FIG. 9B. Upper panel shows results of relative intensities of the Z,Z and 3 photo isomers determined from HPLC scans at multiple times over the 5 hour exposure. This shows that the Z,Z decreases monotonically to low levels while the photoisomers peak at about 1.5 to 2 hours and then decrease towards zero. The bottom panel shows the results of two samples spiked with bilirubin and whose Bf levels were 8 and 12 nM before photo therapy by both Arrows and the Bf assay. In contrast to Arrows, the Bf assay followed the Z,Z monotonic decrease towards zero while the Arrow Bf rose rapidly by 1 hour had reached its saturation level of 50 nM. Thus, Arrows is highly sensitive to photoisomers.

Bleaching serum with a photo therapy lamp as in FIG. 9B reveals that after 5 hours the Bf concentration by UBCheck is effectively zero. Such experiments allow quantitation of the limit of detection, limit of quantitation and in addition they demonstrate that in serum or plasma devoid of bilirubin the UBCheck is unaffected by any other blood containing molecule including other hydrophobic metabolites such as fatty acids, other lipids, peptides, nucleic acids, etc. Importantly, light from the phototherapy lamp is unlikely to be destructive to other blood components given that photo therapy treatment for infants is typically used for much longer times (typically 24 to 72 hours). Not only is the UBCheck insensitive to photoisomers, because the UBCheck's response trends to zero it indicates that the assay is insensitive to all native blood metabolites, including FFA, peptides, nucleic acids, or any other native blood components.

### Example 14

### Lack of Interference from Intralipid and Triglycerides

FIGS. 8A-8B show that intralipid can, as a triglyceride, reduce Bf (FIG. 8) and upon lipolysis produces unbound FFA that can increase Bf by displacing bilirubin from albumin (FIG. 8B). In FIG. 8A, a neonate serum sample spiked with bilirubin to yield a Bf of 24 nM was titrated with triglyceride (Intralipid) up to 10 mM which produced a monotonic decrease in Bf to 18 nM. These results likely correspond to Intralipid infusion without heparin. The decrease in Bf is correctly monitored by the UBCheck methods so that triglycerides produce a sink for bilirubin and is therefore not an interferant. FIG. 8B, shows results for approximately 100 premature infants that received Intralipid infusions, in the presence of heparin, starting with lg/kg/day (IL1) then 2 g/kg/day (IL2) and to 3 g/kg/day (IL3). At each Intralipid step the Bf levels increased because of heparin activated lipase production of unbound FFA (FFAu) which displace bilirubin from albumin. These results are consistent with no interference of triglycerides or FFA on the Bf measurement. In other words turbidity (light scattering) has no effect on the assay, at least for the following reasons: 1) the excitation (660 nm) and emissions (700 and 819 nm) of the fluorescence is in the NIR (light scattering decrease with increasing wavelengths), 2) the light path length of the cartridge is 0.1 mm and 3) if the 700/819 ratio was affected by scattering it would reduce the ratio because the 700 intensity would decrease more than the 819 intensity and thereby decreasing the R value and thereby increasing Bf rather than decreasing Bf as in FIG. 8A. In contrast, the peroxidase method implemented by Arrows, which measurement is performed at 460 nm is highly affected by scattering because Arrows reports an increase in total bilirubin with increasing Intralipid levels.

### Example 15

### Lack of Interference from FFAu

Lipid infusion, for example Intralipid in the presence of heparin, can produce exceptionally high concentrations of unbound FFA (FFAu) much of which displaces bilirubin from albumin (FIG. 4). In neonates receiving Intralipid at increasing concentrations, FFAu levels can increase to over 100 nM [Hegyi, T., et al, Effects of Soybean Lipid Infusion on Unbound Free Fatty Acids and Unbound Bilirubin in Preterm Infants. (2017) J Pediatr 184, 45-50 e41]. However, the results in FIG. 4 reveal that FFA do not interfere with the Bf sensor because at an oleic acid to albumin mole ratio of 6:1, the unbound oleic acid concentration is greater than 500 nM [Richieri et al Interactions of long chain fatty acids and albumin: Determination of free fatty acid levels using the fluorescent probe ADIFAB Biochemistry. 32 7574-7580 (1993)] and yet only increasing bilirubin to albumin affects the sensor by increasing Bf.

### Example 16

### Analytical Specifications and Measurement of Bf in Bilirubin Spiked and Unspiked Human Serum/Plasma

Analytical specifications, following CLSI guidelines, of the UBCheck assay were determined with multiple cartridge lots and 2 or more readers. Results include limit of blank (LOB=0.7 nM) determined by complete bleaching of serum samples, limit of detection (LOD=0.9 nM) and limit of quantitation (LOQ=). A 3-site precision study was performed using 3 readers, 3 different cartridge lots and bilirubin spiked neonatal and adult serum samples. The results as averages of the 3 readers and cartridge lots yielded the following (UB levels in nM and CVs in %): 4.4, 9%; 8.0, 7%; 11.4, 8%; 19.1, 7%; 38.2, 8%).

Pooled human plasma (Golden West Biologicals) for which the concentration of albumin was 620 μM, was spiked with bilirubin to generate plasma samples with bilirubin/albumin mole ratios of approximately 0 to 0.9. Measurements of [Bf] were also performed on the same samples using the peroxidase method implemented in the Arrows Bf Analyzer. These results demonstrate that the response of the bilirubin probes is entirely due to the interaction with bilirubin, the bilirubin probes are not responsive to other metabolites present in human blood samples. Moreover, the agreement with the peroxidase method and with the predictions of bilirubin-albumin equilibrium also demonstrates that the probes yield accurate unbound bilirubin concentrations.

As used herein, the section headings are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose, including the disclosures specifically referenced herein. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein.

In this application, the use of the singular includes the plural unless specifically stated otherwise. Also, the use of “comprise”, “comprises”, “comprising”, “contain”, “contains”, “containing”, “include”, “includes”, and “including” are not intended to be limiting.

As used in this specification and claims, the singular forms “a,” “an” and “the” include plural references unless the content clearly dictates otherwise.

Although this disclosure has been described in the context of certain embodiments and examples, those skilled in the art will understand that the present disclosure extends beyond the specifically disclosed embodiments to other alternative embodiments and/or uses thereof and obvious modifications and equivalents thereof. In addition, while several variations have been shown and described in detail, other modifications, which are within the scope of this disclosure, will be readily apparent to those of skill in the art based upon this disclosure. It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes or embodiments. Thus, it is intended that the scope of the present disclosure herein described should not be limited by the particular disclosed embodiments described above.

It should be understood, however, that this detailed description, while indicating various embodiments, is given by way of illustration only, since various changes and modifications within the spirit and scope thereof will become apparent to those skilled in the art.

The terminology used in the description presented herein is not intended to be interpreted in any limited or restrictive manner. Rather, the terminology is simply being utilized in conjunction with a detailed description of embodiments of the systems, methods and related components. Furthermore, embodiments may comprise several novel features, no single one of which is solely responsible for its desirable attributes or is believed to be essential to practicing the embodiments herein described.

1. A sensor for measuring free bilirubin in a sample, the sensor comprising:
a bilirubin responsive probe labeled with a first fluorophore; and
a non-responder probe labeled with a second fluorophore,
wherein the first and second fluorophores excite at the same wavelength, and
wherein the first and second fluorophore emit a fluorescence at different wavelengths.

2. The sensor of claim 1, wherein the bilirubin responsive probe comprises a first intracellular lipid binding protein (iLBP), wherein the first iLBP has a peptide sequence comprising SEQ ID NO: 1, comprising:
Arg substituting for fourteen accessible lysines (KR14 as set forth in SEQ ID NO: 2);
a C terminal double His tag linker (C2XH11) having a sequence as set forth in SEQ ID NO: 3;
an N terminal addition of MGI; and
no more than 62 amino acid substitutions and additions including a single cysteine.

3. The sensor of claim 1, wherein the bilirubin responsive probe comprise a sequence of any one of the probes as set forth in Table 1.

4. The sensor of claim 1, wherein the non-responder probe comprises a second iLBP, wherein the second iLBP has a peptide sequence comprising SEQ ID NO: 1, comprising:
substitutions at positions 72, 73, 74, 126, and 131;
a substitution to Cys at any one of positions 27, 31, 33, 54, 73, 74, 76, or 98;
no more than three additional amino acid substitutions; and
a C terminal double His tag linker (C2XH11) having a sequence as set forth in SEQ ID NO: 3.

5. The sensor of claim 1, wherein the non-responder probe comprises a sequence of any one of the probes as set forth in Table 2.

6. The sensor of claim 1, wherein the first fluorophore and the second fluorophore are different fluorophores.

7. The sensor of claim 1, wherein the bilirubin responsive probe comprises a single cysteine to which the first fluorophore is attached.

8. The sensor of claim 1, wherein the non-responder probe comprises a single cysteine to which the second fluorophore is attached.

9. The sensor of claim 1, wherein the first fluorophore is LICOR 700DX maleimide or LICOR 800CW maleimide attached to a cysteine substitution.

10. The sensor of claim 1, wherein the bilirubin responsive probe is configured to bind to an unconjugated IX-α (Z,Z) isomer of bilirubin.

11. The sensor of claim 1, wherein the bilirubin responsive probe is configured to minimally bind to conjugated bilirubin (below 4 mg/dl).

12. The sensor of claim 1, wherein the bilirubin responsive probe is configured to not bind to Z,E or E,Z photoisomers of bilirubin, to lumirubin, to fatty acids, to any other naturally occurring blood components, and/or to neonatal drugs.

13. The sensor of claim 12, wherein the neonatal drugs is not spironolactone.

14. The sensor of claim 1, wherein the non-responder probe does not bind to an unconjugated IX-α (Z,Z) isomer of bilirubin or to conjugated bilirubin.

15. The sensor of claim 1, wherein the non-responder probe is configured to not bind to Z,E or E,Z photoisomers of bilirubin, to lumirubin, to fatty acids, to any other naturally occurring blood components, and/or to neonatal drugs.

16. The sensor of claim 1, wherein when the first fluorophore is LICOR 700DX maleimide, the second fluorophore is LICOR 800CW maleimide attached to a cysteine substitution, and when the first fluorophore is LICOR 800CW maleimide, the second fluorophore is LICOR 700DX maleimide.

17. The sensor of claim 1, wherein the first fluorophore or the second fluorophore is attached to a cysteine substitution and wherein the cysteine substitution is at position 22, 24, 25, 26, 27, 29, 30, 33, 54, 74, 76, 97, or 98 of SEQ ID NO: 1.

18. The sensor of claim 1, wherein an emission intensity of the first fluorophore or the second fluorophore is not affected by the absorbance of blood components selected from bilirubin and hemoglobin.

19. The sensor of claim 1, wherein the bilirubin responsive probe or the non-responder probe further comprising at least one linker.

20-24. (canceled)

25. A solid substrate comprising the sensor of claim 1, wherein the bilirubin responsive probe and/or the non-responder probe is attached to the solid substrate.

26. The solid substrate of claim 25, wherein the solid substrate is a Ni-polystyrene, Ni-latex, or Ni-agarose bead.

27. The solid substrate of claim 26, wherein the Ni-polystyrene, Ni-latex, or Ni-agarose bead comprises iron.

28. The solid substrate of claim 25, wherein the bilirubin responsive probe or the non-responder probe comprises substitutions 7R 16R 20R 29R 37R 46R 50R 88R 92R 94R 100R 125R 129R and 130R (KR14) as set forth in SEQ ID NO: 3.

29. The solid substrate of claim 25, wherein the bilirubin responsive probe and/or the non-responder probe comprises a tag and the solid substrate comprises a receptor for the tag.

30. The solid substrate of claim 29, wherein the tag comprises one or more of His-tag, biotin, Flag-epitope, c-myc epitope, HA-tag, glutathione-S-transferase (GST), maltose binding protein (MBP), a chitin binding domain (CBD), Thioredoxin, β-Galactosidase, VSV-Glycoprotein, calmodulin binding protein, a polystyrene (PS) hydrophobic tag, or a metal affinity tag.

31. The solid substrate of claim 29, wherein the tag is a poly-histidine tag and the solid substrate comprises an immobilized metal chelate.

32. The solid substrate of claim 25, wherein the first fluorophore is attached to a cysteine residue on the bilirubin response probe.

33. The solid substrate of claim 25, wherein the second fluorophore is attached to a cysteine residue on the non-responder probe.

34-58. (canceled)


SEQUENCE LISTING (29 sequences)

SEQ ID NO: 1 - protein, 131 aa, Rattus norvegicus
  PEPTIDE (1)...(131): wild-type rat intestinal fatty acid binding protein
  ACETYLATION (1)...(1)
AFDGTWKVDRNENYEKFMEKMGINVVKRKLGAHDNLKLTITQEGNKFTVKESSNFRNIDVVFELGVDFAYSLADGTELTGTWTMEGNKLVGKFKRVDNGKELIAVREISGNELIQTYTYEGVEAKRIFKKE

SEQ ID NO: 2 - protein, 131 aa, Artificial sequence
  arginine substituting for fourteen accessible lysines
AFDGTWRVDRNENYERFMERMGINVVKRRLGAHDNLRLTITQEGNRFTVRESSNFRNIDVVFELGVDFAYSLADGTELTGTWTMEGNRLVGRFRRVDNGRELIAVREISGNELIQTYTYEGVEARRIFRRE

SEQ ID NO: 3 - protein, 28 aa, Artificial sequence
  C terminal double His tag linker
RGAASHHHHHHSHRATPNTSPHHHHHHH

SEQ ID NO: 4 - protein, 162 aa, Artificial sequence
  mutated bf sensitive probe
MGIAFDGTWRVDRNENRERFLERMGICVVARRLGAHDNLRVTITQEGNRFTVRESSNFRNIDRVFELGVDFAYSLFDGTELTGTWTMEGNRLVGRFRRVDNGRELIAVLEISGNELIRTDTYEGVEARRIFRRDRGAASHHHHHHSHRATPNTSPHHHHHHH

SEQ ID NO: 5 - protein, 162 aa, Artificial sequence
  mutated bf sensitive probe
MGIAFDGTWRVDRNENRERFLERMGINCVARRLGAHDNLRVTITQEGNRFTVRESSNFRNIDRVFELGVDFAYSLFDGTELTGTWTMEGNRLVGRFRRVDNGRELIAVLEISGNELIRTDTYEGVEARRIFRRDRGAASHHHHHHSHRATPNTSPHHHHHHH

SEQ ID NO: 6 - protein, 162 aa, Artificial sequence
  mutated bf sensitive probe
MGIAFDGTWRVDRNENRERFLERMGINVCARRLGAHDNLRVTITQEGNRFTVRESSNFRNIDRVFELGVDFAYSLFDGTELTGTWTMEGNRLVGRFRRVDNGRELIAVLEISGNELIRTDTYEGVEARRIFRRDRGAASHHHHHHSHRATPNTSPHHHHHHH

SEQ ID NO: 7 - protein, 162 aa, Artificial sequence
  mutated bf sensitive probe
MGIAFDGTWRVDRNENRERFLERMGINVVCRRLGAHDNLRVTITQEGNRFTVRESSNFRNIDRVFELGVDFAYSLFDGTELTGTWTMEGNRLVGRFRRVDNGRELIAVLEISGNELIRTDTYEGVEARRIFRRDRGAASHHHHHHSHRATPNTSPHHHHHHH

SEQ ID NO: 8 - protein, 162 aa, Artificial sequence
  mutated bf sensitive probe
MGIAFDGTWRVDRNENRERFLERMGINVVARCLGAHDNLRVTITQEGNRFTVRESSNFRNIDRVFELGVDFAYSLFDGTELTGTWTMEGNRLVGRFRRVDNGRELIAVLEISGNELIRTDTYEGVEARRIFRRDRGAASHHHHHHSHRATPNTSPHHHHHHH

SEQ ID NO: 9 - protein, 162 aa, Artificial sequence
  mutated bf sensitive probe
MGIAFDGTWRVDRNENRERFLERMGINVVARRCGAHDNLRVTITQEGNRFTVRESSNFRNIDRVFELGVDFAYSLFDGTELTGTWTMEGNRLVGRFRRVDNGRELIAVLEISGNELIRTDTYEGVEARRIFRRDRGAASHHHHHHSHRATPNTSPHHHHHHH

SEQ ID NO: 10 - protein, 162 aa, Artificial sequence
  mutated bf sensitive probe
MGIAFDGTWRVDRNENRERFLERMGINVVARRLGACDNLRVTITQEGNRFTVRESSNFRNIDRVFELGVDFAYSLFDGTELTGTWTMEGNRLVGRFRRVDNGRELIAVLEISGNELIRTDTYEGVEARRIFRRDRGAASHHHHHHSHRATPNTSPHHHHHHH

SEQ ID NO: 11 - protein, 162 aa, Artificial sequence
  mutated bf sensitive probe
MGIAFDGTWRVDRNENRERFLERMGINVVARRLGAHDNLRVTITQEGNRFTVRESSCFRNIDRVFELGVDFAYSLFDGTELTGTWTMEGNRLVGRFRRVDNGRELIAVLEISGNELIRTDTYEGVEARRIFRRDRGAASHHHHHHSHRATPNTSPHHHHHHH

SEQ ID NO: 12 - protein, 162 aa, Artificial sequence
  mutated bf sensitive probe
MGIAFDGTWRVDRNENRERFLERMGINVVARRLGAHDNLRVTITQEGNRFTVRESSNFRNIDRVFELGVDFAYSLFCGTELTGTWTMEGNRLVGRFRRVDNGRELIAVLEISGNELIRTDTYEGVEARRIFRRDRGAASHHHHHHSHRATPNTSPHHHHHHH

SEQ ID NO: 13 - protein, 162 aa, Artificial sequence
  mutated bf sensitive probe
MGIAFDGTWRVDRNENRERFLERMGINVVARRLGAHDNLRVTITQEGNRFTVRESSNFRNIDRVFELGVDFAYSLFDGCELTGTWTMEGNRLVGRFRRVDNGRELIAVLEISGNELIRTDTYEGVEARRIFRRDRGAASHHHHHHSHRATPNTSPHHHHHHH

SEQ ID NO: 14 - protein, 162 aa, Artificial sequence
  mutated bf sensitive probe
MGIAFDGTWRVDRNENRERFLERMGINVVARRLGAHDNLRVTITQEGNRFTVRESSNFRNIDRVFELGVDFAYSLFDGTELTGTWTMEGNRLVGRFRRVCNGRELIAVLEISGNELIRTDTYEGVEARRIFRRDRGAASHHHHHHSHRATPNTSPHHHHHHH

SEQ ID NO: 15 - protein, 162 aa, Artificial sequence
  mutated bf sensitive probe
MGIAFDGTWRVDRNENRERFLERMGINVVARRLGAHDNLRVTITQEGNRFTVRESSNFRNIDRVFELGVDFAYSLFDGTELTGTWTMEGNRLVGRFRRVDCGRELIAVLEISGNELIRTDTYEGVEARRIFRRDRGAASHHHHHHSHRATPNTSPHHHHHHH

SEQ ID NO: 16 - protein, 159 aa, Artificial sequence
  nonresponder mutated probe
AFDGTWKVDRNENYEKFMEKMGINVVCRKLGAHDNLKLTITQEGNKFTVKESSNFRNIDVVFELGVDFAYSAMFGTELTGTWTMEGNKLVGKFKRVDNGKELIAVREISGNELIQTYTYEGVEAKWIFKKDRGAASHHHHHHSHRATPNTSPHHHHHHH

SEQ ID NO: 17 - protein, 159 aa, Artificial sequence
  nonresponder mutated probe
AFDGTWKVDRNENYEKFMEKMGINVVKRKLCAHDNLKLTITQEGNKFTVKESSNFRNIDVVFELGVDFAYSAMFGTELTGTWTMEGNKLVGKFKRVDNGKELIAVREISGNELIQTYTYEGVEAKWIFKKDRGAASHHHHHHSHRATPNTSPHHHHHHH

SEQ ID NO: 18 - protein, 159 aa, Artificial sequence
  nonresponder mutated probe
AFDGTWKVDRNENYEKFMEKMGINVVKRKLGACDNLKLTITQEGNKFTVKESSNFRNIDVVFELGVDFAYSAMFGTELTGTWTMEGNKLVGKFKRVDNGKELIAVREISGNELIQTYTYEGVEAKWIFKKDRGAASHHHHHHSHRATPNTSPHHHHHHH

SEQ ID NO: 19 - protein, 159 aa, Artificial sequence
  nonresponder mutated probe
AFDGTWKVDRNENYEKFMEKMGINVVKRKLGAHDNLKLTITQEGNKFTVKESSCFRNIDVVFELGVDFAYSAMFGTELTGTWTMEGNKLVGKFKRVDNGKELIAVREISGNELIQTYTYEGVEAKWIFKKDRGAASHHHHHHSHRATPNTSPHHHHHHH

SEQ ID NO: 20 - protein, 159 aa, Artificial sequence
  nonresponder mutated probe
AFDGTWKVDRNENYEKFMEKMGINVVKRKLGAHDNLKLTITQEGNKFTVKESSNFRNIDVVFELGVDFAYSAMCGTELTGTWTMEGNKLVGKFKRVDNGKELIAVREISGNELIQTYTYEGVEAKWIFKKDRGAASHHHHHHSHRATPNTSPHHHHHHH

SEQ ID NO: 21 - protein, 159 aa, Artificial sequence
  nonresponder mutated probe
AFDGTWKVDRNENYEKFMEKMGINVVKRKLGAHDNLKLTITQEGNKFTVKESSNFRNIDVVFELGVDFAYSAMFGCELTGTWTMEGNKLVGKFKRVDNGKELIAVREISGNELIQTYTYEGVEAKWIFKKDRGAASHHHHHHSHRATPNTSPHHHHHHH

SEQ ID NO: 22 - protein, 159 aa, Artificial sequence
  nonresponder mutated probe
AFDGTWKVDRNENYEKFMEKMGINVVKRKLGAHDNLKLTITQEGNKFTVKESSNFRNIDVVFELGVDFAYSAMFGTELTGTWTMEGNKLVGKFKRVDCGKELIAVREISGNELIQTYTYEGVEAKWIFKKDRGAASHHHHHHSHRATPNTSPHHHHHHH

SEQ ID NO: 23 - protein, 159 aa, Artificial sequence
  nonresponder mutated probe
AFDGTWKVDRNENYEKFMEKMGINVVKRKLGAHDNLKLTITQEGNKFTVKESSCFRNIDVVFELGVDFAYSAVFGTELTGTWTMEGNKLVGKFKRVDNGKELIAVREISGNELIQTYTYEGVEAKWIFKKDRGAASHHHHHHSHRATPNTSPHHHHHHH

SEQ ID NO: 24 - protein, 159 aa, Artificial sequence
  nonresponder mutated probe
AFDGTWKVDRNENYEKFMEKMGINVVKRKLGAHDNLKLTITQEGNKFTVKESSNFRNIDVVFELGVDFAYSAVCGTELTGTWTMEGNKLVGKFKRVDNGKELIAVREISGNELIQTYTYEGVEAKWIFKKDRGAASHHHHHHSHRATPNTSPHHHHHHH

SEQ ID NO: 25 - protein, 159 aa, Artificial sequence
  nonresponder mutated probe
AFDGTWKVDRNENYEKFMEKMGINVVKRKLGAHDNLKLTITQEGNKFTVKESSNFRNIDVVFELGVDFAYSAVFGCELTGTWTMEGNKLVGKFKRVDNGKELIAVREISGNELIQTYTYEGVEAKWIFKKDRGAASHHHHHHSHRATPNTSPHHHHHHH

SEQ ID NO: 26 - protein, 159 aa, Artificial sequence
  nonresponder mutated probe
AFDGTWKVDRNENYEKFMEKMGINVVKRKLGAHDNLKLTITQEGNKFTVKESSNFRNIDVVFELGVDFAYSAVFGTELTGTWTMEGNKLVGKFKRVDCGKELIAVREISGNELIQTYTYEGVEAKWIFKKDRGAASHHHHHHSHRATPNTSPHHHHHHH

SEQ ID NO: 27 - protein, 159 aa, Artificial sequence
  nonresponder mutated probe
AFDGTWKVDRNENYEKFMEKMGINVVKRKLGAHDNLKLTITQEGNKFTVKESSNFRNIDVVFELGVDFAYSACFGTELTGTWTMEGNKLVGKFKRVDNGKELIAVREISGNELIQTYTYEGVEAKWIFKKDRGAASHHHHHHSHRATPNTSPHHHHHHH

SEQ ID NO: 28 - protein, 159 aa, Artificial sequence
  nonresponder mutated probe
AFDGTWKVDRNENYEKFMERMGINVVARRLGAHDNLKLTITQEGNKFTVKESSNFRNIDVVFELGVDFAYSACFGTELTGTWTMEGNKLVGKFKRVDNGRELIAVREISGNELIQTYTYEGVEAKWIFKKDRGAASHHHHHHSHRATPNTSPHHHHHHH

SEQ ID NO: 29 - protein, 33 aa, Artificial sequence
  Hb blocking peptide
DTETDEYAASHHHHHHSHRATTPNTSPHHHHHH